SEQUENCE LISTING (54 sequences)

SEQ ID NO: 1 - DNA, 375 bp, Vibrio cholerae
atgattaaattaaaatttggtgttttttttacagttttactatcttcagcatatgcaaatggaacacctcaaaatattactgatttgtgtgcagaataccacaacacacaaatacatacgctaaatgataagatattttcgtatacagaatctctagctggaaaaagagagatggctatcattacttttaagaatggtgcaacttttcaagtagaagtaccaggtagtcaacatatagattcacaaaaaaaagcgattgaaaggatgaaggataccctgaggattgcatatcttactgaagctaaagtcgaaaagttatgtgtatggaataataaaacgcctcatgcgattgccgcaattagtatggcaaattaa

SEQ ID NO: 2 - protein, 124 aa, Vibrio cholerae
MIKLKFGVFFTVLLSSAYANGTPQNITDLCAEYHNTQIHTLNDKIFSYTESLAGKREMAIITFKNGATFQVEVPGSQHIDSQKKAIERMKDTLRIAYLTEAKVEKLCVWNNKTPHAIAAISMAN

SEQ ID NO: 3 - DNA, 26 bp, Artificial Sequence
  Oligonucleotide used in gene assembly cloning
gtttaactttaagaaggagatatacc

SEQ ID NO: 4 - DNA, 25 bp, Artificial Sequence
  Oligonucleotide used in gene assembly cloning
cgaattcgagctccgtcgacaagct

SEQ ID NO: 5 - DNA, 411 bp, Artificial Sequence
  Recombinant fusion containing His tag, linker, antigen, protease recognition, cholera toxin subunit
atgcaccaccatcatcaccatggtggtggctcatggtcacacccacagtttgagaaaggtggcggatcggaaaacctgtatttccaaggtgggggatcgacccctcagaacattaccgatctttgcgcggagtatcataatacccaaattcacacacttaatgacaagattttttcgtacactgagtcacttgcgggaaagcgcgaaatggcgatcatcacctttaagaacggagcgaccttccaggtagaagttcccgggagtcagcacatcgacagccagaagaaagctattgaacgcatgaaagacactttacgcattgcatatttaaccgaggccaaagtggaaaagctgtgcgtgtggaacaacaaaacgcctcacgcaattgcggcaattagcatggctaattaa

SEQ ID NO: 6 - protein, 136 aa, Artificial Sequence
  Recombinant fusion containing His tag, linker, antigen, protease recognition, cholera toxin subunit
MHHHHHHGGGSWSHPQFEKGGGSENLYFQGGGSTPQNITDLCAEYHNTQIHTLNDKIFSYTESLAGKREMAIITFKNGATFQVEVPGSQHIDSQKKAIERMKDTLRIAYLTEAKVEKLCVWNNKTPHAIAAISMAN

SEQ ID NO: 7 - DNA, 345 bp, Artificial Sequence
  Recombinant fusion containing His tag, linker, antigen, cholera toxin subunit
atgcatcaccatcaccatcacggcggcggatccacacctcaaaatattactgatttgtgtgcagaataccacaacacacaaatacatacgctaaatgataagatattttcgtatacagaatctctagctggaaaaagagagatggctatcattacttttaagaatggtgcaacttttcaagtagaagtaccaggtagtcaacatatagattcacaaaaaaaagcgattgaaaggatgaaggataccctgaggattgcatatcttactgaagctaaagtcgaaaagttatgtgtatggaataataaaacgcctcatgcgattgccgcaattagtatggcaaattaa

SEQ ID NO: 8 - protein, 114 aa, Artificial Sequence
  Recombinant fusion containing His tag, linker, antigen, cholera toxin subunit
MHHHHHHGGGSTPQNITDLCAEYHNTQIHTLNDKIFSYTESLAGKREMAIITFKNGATFQVEVPGSQHIDSQKKAIERMKDTLRIAYLTEAKVEKLCVWNNKTPHAIAAISMAN

SEQ ID NO: 9 - DNA, 309 bp, Artificial Sequence
  Codon optimized cholera toxin subunit
actccccaaaatatcacagatttatgcgcagagtaccataatacccaaatccacacgttgaatgacaagattttcagctacacggaatccttggctggtaagcgtgagatggcgattattacgttcaaaaacggcgctacttttcaggttgaagttccgggcagtcaacatatcgactcacagaaaaaagccatcgagcgcatgaaggatactttgcgtatcgcgtatttgactgaagctaaggtggaaaagttgtgcgtctggaacaacaagactccacacgcgattgccgctatttcgatggctaat

SEQ ID NO: 10 - DNA, 54 bp, Artificial Sequence
  Recombinant codon optimized fusion containing linker, His tag
gggggaggttcagggggcggatcgggcggtggctcacaccatcaccaccaccat

SEQ ID NO: 11 - DNA, 363 bp, Artificial Sequence
  Recombinant codon optimized fusion containing cholera toxin subunit, linker, His tag
actccccaaaatatcacagatttatgcgcagagtaccataatacccaaatccacacgttgaatgacaagattttcagctacacggaatccttggctggtaagcgtgagatggcgattattacgttcaaaaacggcgctacttttcaggttgaagttccgggcagtcaacatatcgactcacagaaaaaagccatcgagcgcatgaaggatactttgcgtatcgcgtatttgactgaagctaaggtggaaaagttgtgcgtctggaacaacaagactccacacgcgattgccgctatttcgatggctaatgggggaggttcagggggcggatcgggcggtggctcacaccatcaccaccaccat

SEQ ID NO: 12 - DNA, 63 bp, Escherichia coli
atgaaaaagacagctatcgcgattgcagtggcactggctggtttcgctaccgtagcgcaggcc

SEQ ID NO: 13 - protein, 21 aa, Escherichia coli
MKKTAIAIAVALAGFATVAQA

SEQ ID NO: 14 - DNA, 63 bp, Artificial Sequence
  Mutated E. coli sequence
atggttaagacagctatcgcgattgcagtggcactggctggtttcgctaccgtagcgcaggcc

SEQ ID NO: 15 - protein, 21 aa, Artificial Sequence
  Mutated E. coli sequence
MVKTAIAIAVALAGFATVAQA

SEQ ID NO: 16 - DNA, 66 bp, Erwinia carotovora
atgaaatacctgctgccgaccgctgctgctggtctgctgctcctcgctgcccagccggcgatggcc

SEQ ID NO: 17 - protein, 22 aa, Erwinia carotovora
MKYLLPTAAAGLLLLAAQPAMA

SEQ ID NO: 18 - DNA, 78 bp, Escherichia coli
atgaaaataaaaacaggtgcacgcatcctcgcattatccgcattaacgacgatgatgttttccgcctcggctctcgcc

SEQ ID NO: 19 - protein, 26 aa, Escherichia coli
MKIKTGARILALSALTTMMFSASALA

SEQ ID NO: 20 - DNA, 429 bp, Artificial Sequence
  Recombinant fusion containing mutated E. coli sequence, cholera toxin subunit, linker, His tag
atggttaagacagctatcgcgattgcagtggcactggctggtttcgctaccgtagcgcaggccactccccaaaatatcacagatttatgcgcagagtaccataatacccaaatccacacgttgaatgacaagattttcagctacacggaatccttggctggtaagcgtgagatggcgattattacgttcaaaaacggcgctacttttcaggttgaagttccgggcagtcaacatatcgactcacagaaaaaagccatcgagcgcatgaaggatactttgcgtatcgcgtatttgactgaagctaaggtggaaaagttgtgcgtctggaacaacaagactccacacgcgattgccgctatttcgatggctaatgggggaggttcagggggcggatcgggcggtggctcacaccatcaccaccaccattaa

SEQ ID NO: 21 - protein, 142 aa, Artificial Sequence
  Recombinant fusion containing mutated E. coli sequence, cholera toxin subunit, linker, His tag
MVKTAIAIAVALAGFATVAQATPQNITDLCAEYHNTQIHTLNDKIFSYTESLAGKREMAIITFKNGATFQVEVPGSQHIDSQKKAIERMKDTLRIAYLTEAKVEKLCVWNNKTPHAIAAISMANGGGSGGGSGGGSHHHHHH

SEQ ID NO: 22 - DNA, 432 bp, Artificial Sequence
  Recombinant fusion containing Erwinia sequence, cholera toxin subunit, linker, His tag
atgaaatacctgctgccgaccgctgctgctggtctgctgctcctcgctgcccagccggcgatggccactccccaaaatatcacagatttatgcgcagagtaccataatacccaaatccacacgttgaatgacaagattttcagctacacggaatccttggctggtaagcgtgagatggcgattattacgttcaaaaacggcgctacttttcaggttgaagttccgggcagtcaacatatcgactcacagaaaaaagccatcgagcgcatgaaggatactttgcgtatcgcgtatttgactgaagctaaggtggaaaagttgtgcgtctggaacaacaagactccacacgcgattgccgctatttcgatggctaatgggggaggttcagggggcggatcgggcggtggctcacaccatcaccaccaccattaa

SEQ ID NO: 23 - protein, 143 aa, Artificial Sequence
  Recombinant fusion containing Erwinia sequence, cholera toxin subunit, linker, His tag
MKYLLPTAAAGLLLLAAQPAMATPQNITDLCAEYHNTQIHTLNDKIFSYTESLAGKREMAIITFKNGATFQVEVPGSQHIDSQKKAIERMKDTLRIAYLTEAKVEKLCVWNNKTPHAIAAISMANGGGSGGGSGGGSHHHHHH

SEQ ID NO: 24 - DNA, 444 bp, Artificial Sequence
  Recombinant fusion containing E. coli sequence, cholera toxin subunit, linker, His tag
atgaaaataaaaacaggtgcacgcatcctcgcattatccgcattaacgacgatgatgttttccgcctcggctctcgccactccccaaaatatcacagatttatgcgcagagtaccataatacccaaatccacacgttgaatgacaagattttcagctacacggaatccttggctggtaagcgtgagatggcgattattacgttcaaaaacggcgctacttttcaggttgaagttccgggcagtcaacatatcgactcacagaaaaaagccatcgagcgcatgaaggatactttgcgtatcgcgtatttgactgaagctaaggtggaaaagttgtgcgtctggaacaacaagactccacacgcgattgccgctatttcgatggctaatgggggaggttcagggggcggatcgggcggtggctcacaccatcaccaccaccattaa

SEQ ID NO: 25 - protein, 147 aa, Artificial Sequence
  Recombinant fusion containing E. coli sequence, cholera toxin subunit, linker, His tag
MKIKTGARILALSALTTMMFSASALATPQNITDLCAEYHNTQIHTLNDKIFSYTESLAGKREMAIITFKNGATFQVEVPGSQHIDSQKKAIERMKDTLRIAYLTEAKVEKLCVWNNKTPHAIAAISMANGGGSGGGSGGGSHHHHHH

SEQ ID NO: 26 - protein, 30 aa, Artificial Sequence
  Genetically modified cross-reacting material (CRM197) of diphtheria toxin
SILPGIGSVMGIADGAVHHNTEEIVAQSIA

SEQ ID NO: 27 - DNA, 90 bp, Artificial Sequence
  Codon optimized cross-reacting material (CRM197) of diphtheria toxin
tcaatcttgccgggcattggcagcgtgatgggaatcgctgacggggcggtacaccacaacactgaggagatcgttgcccagtctattgct

SEQ ID NO: 28 - protein, 27 aa, Artificial Sequence
  Genetically modified cross-reacting material (CRM197) of diphtheria toxin
AYNFVESIINLFQVVHNSYNRPAYSPG

SEQ ID NO: 29 - DNA, 81 bp, Artificial Sequence
  Codon optimized cross-reacting material (CRM197) of diphtheria toxin
gcatataattttgtggagtcaattatcaatctttttcaggtggtacacaactcttacaaccgtccagcatacagccccggt

SEQ ID NO: 30 - DNA, 615 bp, Artificial Sequence
  Recombinant fusion containing E. coli sequence, CRM sequence, cholera toxin subunit, CRM sequence, linker, His tag
atgaaaataaaaacaggtgcacgcatcctcgcattatccgcattaacgacgatgatgttttccgcctcggctctcgcctcaatcttgccgggcattggcagcgtgatgggaatcgctgacggggcggtacaccacaacactgaggagatcgttgcccagtctattgctactccccaaaatatcacagatttatgcgcagagtaccataatacccaaatccacacgttgaatgacaagattttcagctacacggaatccttggctggtaagcgtgagatggcgattattacgttcaaaaacggcgctacttttcaggttgaagttccgggcagtcaacatatcgactcacagaaaaaagccatcgagcgcatgaaggatactttgcgtatcgcgtatttgactgaagctaaggtggaaaagttgtgcgtctggaacaacaagactccacacgcgattgccgctatttcgatggctaatgcatataattttgtggagtcaattatcaatctttttcaggtggtacacaactcttacaaccgtccagcatacagccccggtgggggaggttcagggggcggatcgggcggtggctcacaccatcaccaccaccattaa

SEQ ID NO: 31 - protein, 204 aa, Artificial Sequence
  Recombinant fusion E. coli sequence, CRM sequence, cholera toxin subunit, CRM sequence, linker, His tag
MKIKTGARILALSALTTMMFSASALASILPGIGSVMGIADGAVHHNTEEIVAQSIATPQNITDLCAEYHNTQIHTLNDKIFSYTESLAGKREMAIITFKNGATFQVEVPGSQHIDSQKKAIERMKDTLRIAYLTEAKVEKLCVWNNKTPHAIAAISMANAYNFVESIINLFQVVHNSYNRPAYSPGGGGSGGGSGGGSHHHHHH

SEQ ID NO: 32 - protein, 14 aa, Influenza B virus
RGLFGAIAGFIEGG

SEQ ID NO: 33 - DNA, 42 bp, Artificial Sequence
  Codon optimized Influenza B virus sequence
cgcggtctttttggggcaatcgcaggattcatcgaaggagga

SEQ ID NO: 34 - DNA, 486 bp, Artificial Sequence
  Recombinant fusion containing E. coli sequence, Influenza B virus sequence, cholera toxin subunit, linker, His tag
atgaaaataaaaacaggtgcacgcatcctcgcattatccgcattaacgacgatgatgttttccgcctcggctctcgcccgcggtctttttggggcaatcgcaggattcatcgaaggaggaactccccaaaatatcacagatttatgcgcagagtaccataatacccaaatccacacgttgaatgacaagattttcagctacacggaatccttggctggtaagcgtgagatggcgattattacgttcaaaaacggcgctacttttcaggttgaagttccgggcagtcaacatatcgactcacagaaaaaagccatcgagcgcatgaaggatactttgcgtatcgcgtatttgactgaagctaaggtggaaaagttgtgcgtctggaacaacaagactccacacgcgattgccgctatttcgatggctaatgggggaggttcagggggcggatcgggcggtggctcacaccatcaccaccaccattaa

SEQ ID NO: 35 - protein, 161 aa, Artificial Sequence
  Recombinant fusion containing E. coli sequence, Influenza B virus sequence, cholera toxin subunit, linker, His tag
MKIKTGARILALSALTTMMFSASALARGLFGAIAGFIEGGTPQNITDLCAEYHNTQIHTLNDKIFSYTESLAGKREMAIITFKNGATFQVEVPGSQHIDSQKKAIERMKDTLRIAYLTEAKVEKLCVWNNKTPHAIAAISMANGGGSGGGSGGGSHHHHHH

SEQ ID NO: 36 - protein, 32 aa, SARS-CoV-2 virus
GGNYNYLYRLFRKSNLKPFERDISTEIYQAGS

SEQ ID NO: 37 - DNA, 108 bp, Artificial Sequence
  Recombinant codon optimized fusion containing SARS-CoV-2 virus sequence, linker
ggcgggaactataactacctgtaccgtctgttccgtaagtcaaacctgaaaccatttgaacgcgatatctcaacagagatctaccaagccggatccggtgggggctca

SEQ ID NO: 38 - DNA, 66 bp, Artificial Sequence
  Recombinant codon-optimized fusion containing linker, His tag
gggggaggttcagggggcggatcgggcggtggctcacaccatcaccaccaccatcatcatcaccac

SEQ ID NO: 39 - DNA, 564 bp, Artificial Sequence
  Recombinant fusion containing E. coli sequence, SARS-CoV-2 virus sequence, linker, cholera toxin subunit, His tag
atgaaaataaaaacaggtgcacgcatcctcgcattatccgcattaacgacgatgatgttttccgcctcggctctcgccggcgggaactataactacctgtaccgtctgttccgtaagtcaaacctgaaaccatttgaacgcgatatctcaacagagatctaccaagccggatccggtgggggctcaactccccaaaatatcacagatttatgcgcagagtaccataatacccaaatccacacgttgaatgacaagattttcagctacacggaatccttggctggtaagcgtgagatggcgattattacgttcaaaaacggcgctacttttcaggttgaagttccgggcagtcaacatatcgactcacagaaaaaagccatcgagcgcatgaaggatactttgcgtatcgcgtatttgactgaagctaaggtggaaaagttgtgcgtctggaacaacaagactccacacgcgattgccgctatttcgatggctaatgggggaggttcagggggcggatcgggcggtggctcacaccatcaccaccaccatcatcatcaccactaa

SEQ ID NO: 40 - protein, 187 aa, Artificial Sequence
  Recombinant fusion containing E. coli sequence, SARS-CoV-2 virus sequence, linker, cholera toxin subunit, His tag
MKIKTGARILALSALTTMMFSASALAGGNYNYLYRLFRKSNLKPFERDISTEIYQAGSGGGSTPQNITDLCAEYHNTQIHTLNDKIFSYTESLAGKREMAIITFKNGATFQVEVPGSQHIDSQKKAIERMKDTLRIAYLTEAKVEKLCVWNNKTPHAIAAISMANGGGSGGGSGGGSHHHHHHHHHH

SEQ ID NO: 41 - DNA, 108 bp, Artificial Sequence
  Recombinant codon optimized fusion containing linker, SARS-CoV-2 virus sequence
ggtgggggctcaggcgggaactataactacctgtaccgtctgttccgtaagtcaaacctgaaaccatttgaacgcgatatctcaacagagatctaccaagccggatcc

SEQ ID NO: 42 - DNA, 564 bp, Artificial Sequence
  Recombinant fusion containing E. coli sequence, cholera toxin subunit, linker, SARS-CoV-2 virus sequence, His tag
atgaaaataaaaacaggtgcacgcatcctcgcattatccgcattaacgacgatgatgttttccgcctcggctctcgccactccccaaaatatcacagatttatgcgcagagtaccataatacccaaatccacacgttgaatgacaagattttcagctacacggaatccttggctggtaagcgtgagatggcgattattacgttcaaaaacggcgctacttttcaggttgaagttccgggcagtcaacatatcgactcacagaaaaaagccatcgagcgcatgaaggatactttgcgtatcgcgtatttgactgaagctaaggtggaaaagttgtgcgtctggaacaacaagactccacacgcgattgccgctatttcgatggctaatggtgggggctcaggcgggaactataactacctgtaccgtctgttccgtaagtcaaacctgaaaccatttgaacgcgatatctcaacagagatctaccaagccggatccgggggaggttcagggggcggatcgggcggtggctcacaccatcaccaccaccatcatcatcaccactaa

SEQ ID NO: 43 - protein, 187 aa, Artificial Sequence
  Recombinant fusion containing E. coli sequence, cholera toxin subunit, linker, SARS-CoV-2 virus sequence, His tag
MKIKTGARILALSALTTMMFSASALATPQNITDLCAEYHNTQIHTLNDKIFSYTESLAGKREMAIITFKNGATFQVEVPGSQHIDSQKKAIERMKDTLRIAYLTEAKVEKLCVWNNKTPHAIAAISMANGGGSGGNYNYLYRLFRKSNLKPFERDISTEIYQAGSGGGSGGGSGGGSHHHHHHHHHH

SEQ ID NO: 44 - DNA, 132 bp, Artificial Sequence
  Recombinant codon optimized fusion containing linker, SARS-CoV-2 virus sequence
ggtgggggctcaggaggaggctcaggcggcggatcgggcgggaactataactacctgtaccgtctgttccgtaagtcaaacctgaaaccatttgaacgcgatatctcaacagagatctaccaagccggatcc

SEQ ID NO: 45 - DNA, 576 bp, Artificial Sequence
  Recombinant fusion containing E. coli sequence, cholera toxin subunit, linker, SARS-CoV-2 virus sequence, His tag
atgaaaataaaaacaggtgcacgcatcctcgcattatccgcattaacgacgatgatgttttccgcctcggctctcgccactccccaaaatatcacagatttatgcgcagagtaccataatacccaaatccacacgttgaatgacaagattttcagctacacggaatccttggctggtaagcgtgagatggcgattattacgttcaaaaacggcgctacttttcaggttgaagttccgggcagtcaacatatcgactcacagaaaaaagccatcgagcgcatgaaggatactttgcgtatcgcgtatttgactgaagctaaggtggaaaagttgtgcgtctggaacaacaagactccacacgcgattgccgctatttcgatggctaatggtgggggctcaggaggaggctcaggcggcggatcgggcgggaactataactacctgtaccgtctgttccgtaagtcaaacctgaaaccatttgaacgcgatatctcaacagagatctaccaagccggatccgggggaggttcagggggcggatcgggcggtggctcacaccatcaccaccaccattaa

SEQ ID NO: 46 - protein, 191 aa, Artificial Sequence
  Recombinant fusion containing E. coli sequence, cholera toxin subunit, linker, SARS-CoV-2 virus sequence, His tag
MKIKTGARILALSALTTMMFSASALATPQNITDLCAEYHNTQIHTLNDKIFSYTESLAGKREMAIITFKNGATFQVEVPGSQHIDSQKKAIERMKDTLRIAYLTEAKVEKLCVWNNKTPHAIAAISMANGGGSGGGSGGGSGGNYNYLYRLFRKSNLKPFERDISTEIYQAGSGGGSGGGSGGGSHHHHHH

SEQ ID NO: 47 - protein, 24 aa, SARS-CoV-2 virus
GGNYNYLYRLFRKSNLKPFERDIS

SEQ ID NO: 48 - DNA, 108 bp, Artificial Sequence
  Recombinant codon optimized fusion containing linker, SARS-CoV-2 virus sequence
ggtgggggctcaggaggaggctcaggcggcggatcgggcgggaactataactacctgtaccgtctgttccgtaagtcaaacctgaaaccatttgaacgcgatatctca

SEQ ID NO: 49 - DNA, 552 bp, Artificial Sequence
  Recombinant fusion containing E. coli sequence, cholera toxin subunit, linker, SARS-CoV-2 virus sequence, His tag
atgaaaataaaaacaggtgcacgcatcctcgcattatccgcattaacgacgatgatgttttccgcctcggctctcgccactccccaaaatatcacagatttatgcgcagagtaccataatacccaaatccacacgttgaatgacaagattttcagctacacggaatccttggctggtaagcgtgagatggcgattattacgttcaaaaacggcgctacttttcaggttgaagttccgggcagtcaacatatcgactcacagaaaaaagccatcgagcgcatgaaggatactttgcgtatcgcgtatttgactgaagctaaggtggaaaagttgtgcgtctggaacaacaagactccacacgcgattgccgctatttcgatggctaatggtgggggctcaggaggaggctcaggcggcggatcgggcgggaactataactacctgtaccgtctgttccgtaagtcaaacctgaaaccatttgaacgcgatatctcagggggaggttcagggggcggatcgggcggtggctcacaccatcaccaccaccattaa

SEQ ID NO: 50 - protein, 183 aa, Artificial Sequence
  Recombinant fusion containing E. coli sequence, cholera toxin subunit, linker, SARS-CoV-2 virus sequence, His tag
MKIKTGARILALSALTTMMFSASALATPQNITDLCAEYHNTQIHTLNDKIFSYTESLAGKREMAIITFKNGATFQVEVPGSQHIDSQKKAIERMKDTLRIAYLTEAKVEKLCVWNNKTPHAIAAISMANGGGSGGGSGGGSGGNYNYLYRLFRKSNLKPFERDISGGGSGGGSGGGSHHHHHH

SEQ ID NO: 51 - protein, 14 aa, SARS-CoV-2 virus
GGNYNYLYRLFRKS

SEQ ID NO: 52 - DNA, 78 bp, Artificial Sequence
  Recombinant codon-optimized fusion containing linker, SARS-CoV-2 virus sequence
ggtgggggctcaggaggaggctcaggcggcggatcgggcgggaactataactacctgtaccgtctgttccgtaagtca

SEQ ID NO: 53 - DNA, 522 bp, Artificial Sequence
  Recombinant fusion containing E. coli sequence, cholera toxin subunit, linker, SARS-CoV-2 virus sequence, His tag
atgaaaataaaaacaggtgcacgcatcctcgcattatccgcattaacgacgatgatgttttccgcctcggctctcgccactccccaaaatatcacagatttatgcgcagagtaccataatacccaaatccacacgttgaatgacaagattttcagctacacggaatccttggctggtaagcgtgagatggcgattattacgttcaaaaacggcgctacttttcaggttgaagttccgggcagtcaacatatcgactcacagaaaaaagccatcgagcgcatgaaggatactttgcgtatcgcgtatttgactgaagctaaggtggaaaagttgtgcgtctggaacaacaagactccacacgcgattgccgctatttcgatggctaatggtgggggctcaggaggaggctcaggcggcggatcgggcgggaactataactacctgtaccgtctgttccgtaagtcagggggaggttcagggggcggatcgggcggtggctcacaccatcaccaccaccattaa

SEQ ID NO: 54 - protein, 173 aa, Artificial Sequence
  Recombinant fusion containing E. coli sequence, cholera toxin subunit, linker, SARS-CoV-2 virus sequence, His tag
MKIKTGARILALSALTTMMFSASALATPQNITDLCAEYHNTQIHTLNDKIFSYTESLAGKREMAIITFKNGATFQVEVPGSQHIDSQKKAIERMKDTLRIAYLTEAKVEKLCVWNNKTPHAIAAISMANGGGSGGGSGGGSGGNYNYLYRLFRKSGGGSGGGSGGGSHHHHHH

(19) United States
(12) Patent Application Publication
(10) Pub. No.: US 2022/0273790 A1
(43) Pub. Date: Sep. 1, 2022

(54) RATIONALLY ENGINEERED CARRIER PROTEINS FOR VACCINES

(71) Applicant: Citranvi Biosciences, LLC, Chapel Hill, NC (US)

(72) Inventor: Avvari Krishna Prasad, Chapel Hill, NC (US)

(73) Assignee: Citranvi Biosciences, LLC, Chapel Hill, NC (US)

(21) Appl. No.: 17/618,122

(22) PCT Filed: Jun. 17, 2020
(86) PCT No.: PCT/US2020/038185

§ 371 Date: Dec. 10, 2021

Related U.S. Application Data
(60) Provisional application No. 62/862,916, filed on Jun. 18, 2019.

(51) Int. Cl. A61K 39/385; A61K 39/39; A61K 39/215

(52) U.S. Cl. CPC A61K 39/385; A61K 39/39; A61K 39/215; A61K 2039/645

(57) ABSTRACT

The invention relates to the design of rationally engineered Carrier Proteins (reCaPs) geared towards producing Multifunctional Chimeric recombinant Fusion Proteins (MCFPs) useful as vaccine candidates. The key components of the MCFPs are (i) genetically engineered carrier proteins; (ii) polypeptide antigens; (iii) linker peptides, optionally fused to heterologous T-cell epitopes; (iv) Dual Function Peptides (DFP) which can act as a purification aids as well having the non-covalent affinity to bind to an adjuvant. The present invention also relates to recombinantly expressed Self-Assembling Adjuvanted Nanoparticles (SAANPs), comprising reCaPs fused with various polypeptide and protein antigens, useful as vaccine candidates. The present invention also provides novel ‘integrated Multiple Conjugate Antigen displayed Adjuvanted Systems’ [iMCAAS], comprising rationally engineered Carrier Proteins, based on ‘Self Assembling Adjuvanted Nanoparticles’ [SAANPs]. These adjuvanted nanoparticles, eventually provide stronger antigen-antibody interactions compared to the low affinity interactions provided by the monovalent binding generated by single antigen immunogens.

### CROSS-REFERENCE TO RELATED APPLICATIONS

The present application claims the benefit of priority to U.S. Provisional Application No. 62/862,916, filed Jun. 18, 2019, the contents of which are incorporated herein by reference.

### SEQUENCE LISTING

The instant application contains a Sequence Listing which has been submitted in ASCII format via EFS-Web and is hereby incorporated by reference in its entirety. Said ASCII copy, created on Jun. 17, 2020, is named 12063-002WO1-SEQ_LIST_ST25.txt and is 22 kilobytes in size.

### BACKGROUND

Stimulation of protective immune responses by antigens is the basis for effective prophylactic vaccines against pathogenic microorganisms. Not all antigens induce an appropriate immune response, however. Development of new and improved systems for stimulating protective immune responses is an active area.

### SUMMARY

The invention relates to the design and development of rationally engineered Carrier Proteins (reCaPs) geared towards producing Multifunctional Chimeric recombinant Fusion Proteins (MCFPs) useful as vaccine candidates. The key components of the MCFPs are (i) genetically engineered carrier proteins; (ii) polypeptide antigens; (iii) linker peptides, optionally fused to heterologous T-cell epitopes; (iv) Dual Function Peptides (DFP) which can act as a purification aids as well having the non-covalent affinity to bind to an adjuvant. The present invention also relates to recombinantly expressed Self-Assembling Adjuvanted Nanoparticles (SAANPs), comprising reCaPs fused with various polypeptide and protein antigens, useful as vaccine candidates. The present invention also relates to conjugate vaccine technologies related to design and development of novel reCaPs for immunogenicity enhancement and/or stability enhancement and as vaccine candidates. The invention also relates to the design and development of reCaPs, with no lysine residues in the T cell epitopes, present in the carrier proteins, in order to prevent conjugation of the target antigen to the T-cell epitope regions. The invention also relates to the design and development of reCaPs, with fewer lysine residues, geared towards producing conjugate vaccines with minimal cross-linking. The present invention also relates to the production of Self-Assembling Adjuvanted Nanoparticles (SAANPs), comprising reCaPs chemically conjugated with various antigens. More specifically, the present invention relates to the design and production of SAANPs, comprising MCFPs as well as carrier proteins conjugated with various antigens, to minimize non-specific interactions between the T-cell epitopes of the carrier proteins as well as the critical immunogenic epitopes of the conjugated antigens and the adjuvants.

### BRIEF DESCRIPTION OF THE DRAWINGS

In the accompanying drawings, which are incorporated in and constitute a part of the specification, embodiments of the disclosed inventions are illustrated. It will be appreciated that the embodiments illustrated in the drawings are shown for purposes of illustration and not for limitation. It will be appreciated that changes, modifications and deviations from the embodiments illustrated in the drawings may be made without departing from the spirit and scope of the invention, as disclosed below.

FIG. 1 depicts the current classical vaccine formulation process of the mixing of the conjugated antigen (drug substance) with the adjuvant to produce the “drug product”.

FIG. 2 provides the production design of an example Multifunctional Chimeric Fusion. The recombinantly expressed fusion protein (MCFP) is comprised of the following components: (A) a carrier protein; (B) linker peptide; (C) polypeptide antigen and (D) Dual Function Peptide. The molecule on the left, that contains the A, B, C and D components, is an example of a MCFP. Multimerization, by in situ self-assembly of the MCFPs results in the labeled Drug Substance. Formulation of the Drug Substance with an adjuvant results in the Drug Product. Multimerization of the MCFPs (indicated as “Drug Substance” in the drawing) with adjuvants results in a Drug Product, which is an example of a Self-Assembling Adjuvanted Nanoparticle (SAANP).

FIG. 3 is the cartoon representation of the structure of CTRNV5 fusion protein pentamer, depicting CTB with each of the monomer units attached via a linker to the DFP at the C-terminus. CTB pentamer is represented by ribbons, and the fusing of each of the five monomers units with each of the five (i) linkers and (ii) DFPs, respectively, are shown in stick representation

FIG. 4 is an expression and solubility assessment of CTRNV5. Samples were collected prior to induction with IPTG (denoted with “0”) and at three hours post-induction with IPTG (denoted with “3”). Protein expression was assessed in whole cell protein samples (denoted with “WC”), and solubility of the recombinant protein was assessed in post-induction samples, where “S” indicates the soluble protein fraction and “I” indicates the insoluble protein fraction. Sizes of relevant molecular weight standards are shown at the left of the figure, and the CTRNV5 protein is indicated with a solid arrow at the right of the figure.

FIG. 5 includes data indicating periplasmic localization of CTRNV5. Periplasmic proteins were purified from E. coli by cold osmotic shock. M: Protein molecular weight standard; 1: Whole cell protein from cells prior to induction with IPTG; 2: Whole cell protein from cells post-induction with IPTG; 3: Periplasmic proteins purified by cold osmotic shock. Sizes of relevant molecular weight standards are shown at the left of the figure. CTRNV5 protein is indicated with a solid arrow at the right of the figure.

FIGS. 6A and 6B include data showing purification of CTRNV5. CTRNV5 was purified from the soluble protein fraction by IMAC in batch mode. The progression of purification was followed throughout the procedure by SDS-PAGE. FIG. 6A. M: Protein molecular weight standard; 1: Whole cell protein from cells prior to induction with IPTG; 2: Whole cell protein from cells post-induction with IPTG; 3: Proteins not bound during incubation with IMAC resin; 4: Proteins removed by first wash in buffer containing 5 mM imidazole; 5: Proteins removed by second wash in buffer containing 5 mM imidazole; 6: Proteins removed by first wash in buffer containing 60 mM imidazole; 7: Proteins removed by second wash in buffer containing 60 mM imidazole; 8: Proteins removed by third wash in buffer containing 60 mM imidazole. FIG. 6B. M: Protein molecular weight standard; 1: Whole cell protein from cells prior to induction with IPTG; 2: Whole cell protein from cells post-induction with IPTG; 3: Proteins eluted from the resin from the first wash in buffer containing 1 M imidazole; 4: Proteins eluted from the resin from the second wash in buffer containing 1 M imidazole; 5: Proteins eluted from the resin from the third wash in buffer containing 1 M imidazole; 6: Proteins eluted from the resin from the fourth wash in buffer containing 1 M imidazole; 7: Proteins eluted from the resin from the fifth wash in buffer containing 1 M imidazole. Sizes of relevant molecular weight standards are shown at the left of each panel. CTRNV5 protein is indicated with a solid arrow at the right in FIG. 6B.

FIG. 7 includes data showing recognition of CTRNV5 by a cholera toxin subunit B (CTB)-specific monoclonal antibody in Western blot hybridization. Two identical gels were run; one was stained with Coomassie blue gel stain (Panel A), and the other was transferred to PVDF membrane for Western blot hybridization (Panel B). Panel A. M: Protein molecular weight standard; 1: 1 μg CTB (Sigma-Aldrich); 2: 1 μg purified CTRNV5 protein . Panel B. M: Protein molecular weight standard; 1: 1 μg cholera toxin subunit B (Sigma-Aldrich); 2: 1 μg purified CTRNV5 protein.

FIG. 8 includes data showing binding of GM1 ganglioside by CTRNV5 as assessed by ELISA. Binding of GM1 ganglioside by CTRNV5 was assessed with commercially available cholera toxin subunit B (Sigma CTB, Sigma-Aldrich) used as a positive control. Bovine serum albumin (BSA) was used as a negative control. Wells containing GM1 ganglioside were coated with 100 ng GM1 ganglioside. Some wells in the plate were left uncoated with GM1 ganglioside to ensure assay specificity. Closed circles: Dilution series (0.05-50 ng/well) of CTRNV5 protein in GM1 ganglioside coated wells; Squares: Dilution series (0.05-50 ng/well) of CTRNV5 protein in GM1 ganglioside non-coated wells; Triangles: Dilution series (0.05-50 ng/well) of Sigma CTB protein in GM1 ganglioside coated wells; Inverted triangles: Dilution series (0.05-50 ng/well) of Sigma CTB protein in GM1 ganglioside non-coated wells; Diamonds: Dilution series (0.05-50 ng/well) of BSA protein in GM1 ganglioside coated wells; Open circles: Dilution series (0.05-50 ng/well) of BSA protein in GM1 ganglioside non-coated wells.

FIG. 9 is a cartoon representation of the structure of the CTRNV10 fusion protein CTB-GGGS-SARS-Cov-2 Receptor Binding Domain (RBD) polypeptide-(GGGS)3-His10 at the C-terminus. The CTB pentamer is represented by ribbons, and each of five monomers of the C-terminals fusing with each of the (i) RBD sequences, (ii) linkers and (iii) 10xHis tags, respectively, are shown in stick representation.

FIGS. 10A and 10 B show data for purification of CTRNV11. CTRNV11 was purified from the soluble protein fraction by IMAC in batch mode. The progression of purification was followed throughout the procedure by SDS-PAGE. FIG. 10A. M: Protein molecular weight standard; 1: Whole cell protein from cells prior to induction with IPTG; 2: Whole cell protein from cells post-induction with IPTG; 3: Proteins not bound during incubation with IMAC resin; 4: Proteins removed by first wash in buffer containing 5 mM imidazole; 5: Proteins removed by second wash in buffer containing 5 mM imidazole; 6: Proteins removed by first wash in buffer containing 60 mM imidazole; 7: Proteins removed by second wash in buffer containing 60 mM imidazole; 8: Proteins removed by third wash in buffer containing 60 mM imidazole. FIG. 10B. M: Protein molecular weight standard; 1: Whole cell protein from cells prior to induction with IPTG; 2: Whole cell protein from cells post-induction with IPTG; 3: Proteins eluted from the resin from the first wash in buffer containing 1 M imidazole; 4: Proteins eluted from the resin from the second wash in buffer containing 1 M imidazole; 5: Proteins eluted from the resin from the third wash in buffer containing 1 M imidazole; 6: Proteins eluted from the resin from the fourth wash in buffer containing 1 M imidazole. Sizes of relevant molecular weight standards are shown at the left of each panel. CTRNV11 protein is indicated with a solid arrow at the right in FIG. 10B.

### DETAILED DESCRIPTION

### Definitions

As used herein, the singular forms “a,” “an,” and “the,” refer to both the singular as well as plural, unless the context clearly indicates otherwise. For example, the term “an antigen” or “immunogen” includes single or plural antigens or immunogens and can be considered equivalent to the phrase “at least one antigen” or “at least one immunogen”.

The term “adjuvant” refers to a substance capable of enhancing, accelerating, or prolonging the body's immune response to an immunogen or immunogenic composition, such as a vaccine (although it is not immunogenic by itself). An adjuvant may be included in the immunogenic composition, such as a vaccine.

The term “adjuvanted nanoparticle” means a nanoparticle which is associated with an adjuvant, in some examples, via non-covalent interactions, such as ionic, Hydrogen, hydrophobic, van der Waals forces, etc.

The term “administration” refers to the introduction of a substance or composition into a subject by a chosen route. Administration can be local or systemic. For example, if the chosen route is intramuscular, the composition (such as a composition including a disclosed immunogen) is administered by introducing the composition into a muscle of the subject.

The term “antigen” refers to a molecule that can be recognized by an antibody. Examples of antigens include polypeptides, peptides, lipids, polysaccharides, and nucleic acids containing antigenic determinants, such as those recognized by an immune cell.

The term “blocking core adjuvant surface space from non-specific binding” means the adjuvant surface binds preferentially to a peptide attached to the carrier protein (such as the dual function peptide), in some examples via non-covalent interactions, and minimizes the direct interactions between the antigen (portion of the MCFP) and the adjuvant. The main objective of the blocking is particularly to minimize the direct interactions between the immunogenic epitopes of the antigen and the adjuvant.

The term “bound to” refers to the association of two different molecules via covalent or non-covalent interactions.

The term “carrier protein” refers to a protein that helps an antigen to augment its immunogenic properties. For example, the immunogenicity of small molecules, saccharides and peptides as human vaccines is enhanced by coupling of the antigens to carrier proteins.

The term “Carrier Protein Displayed Adjuvant system” (CAPDAdjuvant system) refers to a macromolecular system that can direct the carrier protein to bind with an adjuvant, in an ordered fashion, to display the antigen (attached to the carrier protein) with the aid of a peptide (such as the DFP, attached to the carrier). The CAPDAdjuvant is designed to minimize the interactions (or association) between the antigen portion and the adjuvant.

The term “chimeric fusion protein” (CFP) refers to a hybrid protein produced by recombinant protein expression via the translation of a fusion gene which results in multiple polypeptides with functional properties derived from one or more parent proteins.

The term “controlling the density of the peptide antigen” means the ability of the adjuvant to associate with multiple copies of the MCFP (via the DFP, specifically) in ordered fashion, in some examples, via non-covalent interactions.

The term “domain” refers to a polypeptide sequence that can evolve, function, and exist independently of the rest of the protein chain. Each domain forms a compact three-dimensional structure and often can be independently stable and folded.

The term “dual function peptide” (DFP) refers to a peptide domain that aids in protein purification as well as having the affinity to bind to adjuvants, in some examples, via non-covalent interactions.

The term “effective amount” refers to an amount of agent that is sufficient to generate a desired response. For instance, this can be the amount necessary to inhibit viral replication or to measurably alter outward symptoms of the viral infection.

The term “epitope” (or “antigenic determinant” or “antigenic site”) refers to the region of an antigen to which an antibody, B cell receptor, or T cell receptor binds or responds. Epitopes can be formed from contiguous amino acids or noncontiguous amino acids juxtaposed by secondary, tertiary, or quaternary folding of a protein. Epitopes formed from contiguous amino acids are typically retained on exposure to denaturing solvents whereas epitopes formed by higher order folding are typically lost on treatment with denaturing solvents.

The term “heterologously derived” refers to incorporation of a sequence, either nucleotide or amino acid, that is not naturally present in a sequence of interest. Incorporation of heterologous sequences can be accomplished, for example, by recombinant DNA technology.

The term “host cells” refers to cells in which a vector can be propagated and its DNA or RNA expressed. The cell may be prokaryotic or eukaryotic.

In another aspect, the present invention provides nucleic acid molecules that encode peptide-linked protein immunogens described herein above. These nucleic acid molecules include DNA, cDNA, and RNA sequences. The nucleic acid molecule can be incorporated into a vector, such as an expression vector.

The term “identical” or percent “identity,” in the context of two or more nucleic acid or polypeptide sequences, refers to two or more sequences or subsequences that are the same or have a specified percentage of nucleotides or amino acid residues or nucleotides that are the same, when compared and aligned for maximum correspondence. Methods of alignment of sequences for comparison are well known in the art. Once aligned, the number of matches is determined by counting the number of positions where an identical nucleotide or amino acid residue is present in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a peptide sequence that has 150 matches when aligned with a test sequence having 300 amino acids is 50.0 percent identical to the test sequence (150/300×100=50.0).

In some examples, the sequences disclosed and/or claimed herein may be 60, 65, 70, 75, 80, 85, 86, 87, 88, 89, 90, 91, 92, 93, 94, 95, 96, 97, 98 or 99% identical to the actual sequences in any of the SEQ ID NOs that are part of this application.

In some examples, the sequences disclosed and/or claimed herein may be at least 60, 65, 70, 75, 80, 85, 86, 87, 88, 89, 90, 91, 92, 93, 94, 95, 96, 97, 98 or 99% identical to the actual sequences in any of the SEQ ID NOs that are part of this application.

In some examples, the sequences disclosed and/or claimed herein may be less than 85, 86, 87, 88, 89, 90, 91, 92, 93, 94, 95, 96, 97, 98 or 99% identical to the actual sequences in any of the SEQ ID NOs that are part of this application.

In some examples, the sequences disclosed herein may be or may be 60, 65, 70, 75, 80, 85, 86, 87, 88, 89, 90, 91, 92, 93, 94, 95, 96, 97, 98 or 99% identical or at least 60, 65, 70, 75, 80, 85, 86, 87, 88, 89, 90, 91, 92, 93, 94, 95, 96, 97, 98 or 99% identical, and less than 85, 86, 87, 88, 89, 90, 91, 92, 93, 94, 95, 96, 97, 98 or 99% identical, as appropriate, to the actual sequences in any of the SEQ ID NOs that are part of this application.

Optimal alignment of sequences for comparison can be conducted using known algorithms.

The term “immunogen” refers to a compound, composition, or substance that is immunogenic as defined herein below.

The term “immunogenic” refers to the ability of a substance to cause, elicit, stimulate, or induce an immune response against a particular antigen, in a subject, whether in the presence or absence of an adjuvant.

The term “immunogenic” refers to the ability of a foreign substance, such as an antigen, to provoke an immune response in the body of a human or other animal, whether in the presence or absence of an adjuvant.

The term “immune response” refers to any detectable response of a cell or cells of the immune system of a host mammal to a stimulus (such as an immunogen), including, but not limited to, innate immune responses (e.g., activation of Toll receptor signaling cascade), cell-mediated immune responses (e.g., responses mediated by T cells, such as antigen-specific T cells, and non-specific cells of the immune system), and humoral immune responses (e.g., responses mediated by B cells, such as generation and secretion of antibodies into the plasma, lymph, and/or tissue fluids). Examples of immune responses include an alteration (e.g., increase) in Toll-like receptor activation, lymphokine (e.g., cytokine (e.g., Th1, Th2 or Th17 type cytokines) or chemokine) expression or secretion, macrophage activation, dendritic cell activation, T cell (e.g., CD4+ or CD8+ T cell) activation, NK cell activation, B cell activation (e.g., antibody generation and/or secretion), binding of an immunogen (e.g., antigen; e.g., immunogenic polypeptide) to an MHC molecule, induction of a cytotoxic T lymphocyte (“CTL”) response, induction of a B cell response (e.g., antibody production), and, expansion (e.g., growth of a population of cells) of cells of the immune system (e.g., T cells and B cells), and increased processing and presentation of antigen by antigen presenting cells. The term “immune response” also encompasses any detectable response to a particular substance (such as an antigen or immunogen) by one or more components of the immune system of a vertebrate animal in vitro.

The term “immunogenic composition” refers to a composition comprising an immunogen.

The term “infectious disease” refers to a disease (such as influenza, malaria, meningitis, rabies, or tetanus) caused by the entrance into the body of pathogenic agents or microorganisms (such as bacteria, viruses, protozoans, or fungi) which grow and multiply there.

The term “integrated Multiple Antigen displayed Adjuvant System” (iMAAS) refers to a set of macromolecules that can direct them to bind with an adjuvant, via non-covalent interactions in an ordered fashion to display the antigen (covalently bound to the carrier protein) with the aid of a peptide (covalently attached to the carrier) such as the DFP. The iMAAS is designed to minimize the direct interactions between the immunogenic epitopes of an antigen and an adjuvant.

The term “integrated Multiple Conjugated Antigen displayed Adjuvant Systems” (iMCAAS) refers to a set of macromolecules that can direct them to bind with an adjuvant, via non-covalent interactions in an ordered fashion to display the conjugated antigen (that is chemically bound to the carrier protein via one or more covalent bonds) with the aid of a peptide (covalently attached to the carrier) such as the DFP. The iMCAAS is designed to minimize the direct interactions between the immunogenic epitopes of a chemically conjugated antigen and an adjuvant.

The term “linker peptide” refers to small peptides that connect protein and polypeptide subunits, and also provide many other functions, such as maintaining cooperative inter-domain interactions or preserving biological activity. Peptide linkers in multi-domain proteins are helpful for the rational design of recombinant fusion proteins. Similar to recombinant fusion proteins, several naturally-occurring multi-domain proteins are composed of two or more functional domains joined by linker peptides.

The term “macromolecule” refers to molecules containing a very large number of atoms, such as lipids, oligosaccharides, polysaccharides, polypeptides, proteins, nucleic acids, or synthetic polymers.

The term “native” or “wild-type” protein, sequence, or polypeptide refers to a naturally existing protein, sequence, or polypeptide that has not been artificially modified by selective mutations.

The term “Multifunctional Chimeric Recombinant Fusion Proteins” (MCFPs) refers to a hybrid protein produced by recombinant protein expression via the translation of a fusion gene which results in multiple polypeptides with functional properties derived from one or more parent proteins. MCFPs are created artificially by recombinant DNA technology. The key components of the MCFPs are (i) genetically engineered carrier proteins; (ii) polypeptide antigens; (iii) linker peptides, optionally fused to heterologous T-cell epitopes; (iv) Dual Function Peptides (DFP) which can perform as purification aids as well possessing the non-covalent affinity to bind to an adjuvant.

The term “nanoparticle” refers to macromolecules or particles of any shape with dimensions in the 1×10⁻⁹ and 1×10⁻⁷ m range. Nanoparticles occur widely in nature and are objects of study in many disciplines such as biology, chemistry and materials science. The production of nanoparticles with specific properties is an important branch of these disciplines. Nanoparticles are used in a number of biomedical applications such as drug carriers. Nanoparticle technology allows for controlled delivery of some drugs to achieve the most desirable biological outcome, example in cancer therapeutics. Some other medical applications include special materials for wound dressings, materials used in implants, tissue engineering, etc.

The term “non-specific binding” means an inter-molecular interaction that is undesirable. For example, the desirable goal of immunogenic epitopes of antigens is to help them interact with specific cells of the immune system such as B-cells. Human B cells have B cell receptors on their surface, which they use to bind to specific immunogenic epitopes of protein antigens. Once the B cells bind to this protein, they release antibodies that stick to the antigen and prevent it from harming the body. Any interaction that interferes or reduces the interactions of the antigens with the B-cells of the immune system is considered as undesirable since it can diminish the specific immunogenic response and reduce vaccine potency.

The term “pharmaceutically acceptable carriers” refers to a material or composition which, when combined with an active ingredient, is compatible with the active ingredient and does not cause toxic or otherwise unwanted reactions when administered to a subject, particularly a mammal. Examples of pharmaceutically acceptable carriers include solvents, surfactants, suspending agents, buffering agents, lubricating agents, emulsifiers, absorbants, dispersion media, coatings, and stabilizers.

The term “pathogenic” refers to causing or capable of causing disease

The term “pathogen-specific” refers to the antigen epitopes that are derived from bacteria or viruses that are specific causative agents of a variety of infectious diseases.

The term “polypeptide antigen” refers to a polypeptide molecule, derived from pathogenic bacteria or viruses, that can be recognized by an antibody. Polypeptide antigens are recognized by immune cells such as antigen presenting cells or B cells.

The term “rationally engineered Carrier Protein” (reCaP) refers to a recombinantly expressed chimeric fusion protein (CFP) that has been designed to engineer additional peptides, such as linkers and/or T cell epitopes into a carrier protein that can further augment an antigen to boost its immunogenic responses, via self-assembly to form nanoparticles, recruit additional T-cell help, etc. The newly engineered T cell epitopes, in some examples, without lysine residues, can stay unmodified during the process of chemical conjugation thereby remain unhindered during the process of soliciting T cell help.

The term “Self Assembling Adjuvanted Nanoparticles” (SAANPs) refers to nanoparticles that are capable of spontaneous organization of smaller peptide domains, without any catalyst, to form larger well-organized patterns together with adjuvants to further augment an immunogenic response relative to soluble proteins that do not have the capability of self assembly.

The term “self assembling” refers to the spontaneous organization of smaller subunits, without any catalyst, to form larger, well-organized patterns. For nanoparticles, this spontaneous assembly is a consequence of interactions between the particles aimed at achieving an equilibrium.

The term “subject” refers to either a human or a non-human mammal. The term “mammal” refers to any animal species of the Mammalia class. Examples of mammals include: humans; non-human primates such as monkeys; laboratory animals such as rats, mice, guinea pigs; domestic animals such as cats, dogs, rabbits, cattle, sheep, goats, horses, and pigs; and captive wild animals such as lions, tigers, elephants, and the like.

The term “vaccine” refers to a pharmaceutical composition comprising an immunogen that is capable of eliciting a prophylactic or therapeutic immune response in a subject. Typically, a vaccine elicits an antigen-specific immune response to an antigen of a pathogen, for example a viral pathogen.

### Vaccine Systems

Even though pathogenic microorganisms contain a number of antigenic components such as proteins, glycoproteins, capsular polysaccharides, lipopolysaccharides, etc., only a selected few of these will induce the appropriate immune response. Therefore, the selection of the immunogen, multi-faceted design criteria, stability and viable manufacturing process criteria along with the delivery strategy in the form of an optimal vaccine construct are essential considerations for the development of an effective vaccine product [Newman et al., 1995; Prasad, 2018].

Many of the currently commercially licensed vaccines typically comprise an antigen (immunogen) and an adjuvant. In the case of “multi-valent vaccines” targeted against microbial diseases, the product contains antigens (immunogens) from many serotypes of the microorganism and optionally, an adjuvant. In the case of “multi-component vaccines”, the product contains antigens (immunogens) from many serotypes of the microorganism as well as one or more heterogeneous antigens (immunogens) and an adjuvant. Antigens are usually macromolecules, such as peptides, carbohydrates, proteins etc., having epitopes (specific antigenic sites) that recognize, interact and bind with various components of the immune systems, such as B lymphocytes. Typically, antigens are perceived by immune systems of living organisms as being foreign, toxic or dangerous and produce antibody molecules to combat these “foreign” antigens. Some antigens such as short peptides, small molecule haptens, some proteins and carbohydrates elicit a poor immunogenic response. One of the modes of enhancing the immunogenicity of small molecule haptens and carbohydrate antigens, in order to be successfully deployed as human vaccines, is by the chemical coupling to carrier proteins [Svenson et al., 1979; Prasad et al., 2018]. This approach to address the poor antibody response of a number of microbial antigens is through the conjugation of the respective antigens to carrier proteins to elicit a more robust immunogenic response. In this type of immune response, elicited by the antigens conjugated to carrier proteins, antibodies are produced and secreted by the B-lymphocytes in conjunction with the T-helper (Th) cells. Using a synergistic approach, the B and T cells synchronize to induce an antigen specific antibody response that is robust and long-lasting memory. This strategy of using conjugation technology has transformed the first generation of T-cell independent purely carbohydrate-based vaccines to the second generation of T cell-dependent conjugate vaccines that are much more immunogenic and launched a renaissance in vaccinology [Dintzis, 1992; Prasad et al., 2018].

Peptide antigens used to generate site-specific antibodies to proteins have been described in the art towards the development of vaccines. The poor immunogenic response of a short peptide could also be amplified by Multiple Antigen Peptide (MAP) based systems [Tam et al., 1988; Posnett, et al., 1988; Posnett, et al.,1989] to overcome the need for chemical conjugation to the carrier protein, forming nanoparticles. In this MAP system, multiple copies of antigenic peptides are simultaneously bound to the α- and ϵ-amino groups of a non-immunogenic Lys-based dendritic scaffold.

The MAP system allows the formation of arrays of a wide range of peptidic nanoparticles in a controlled fashion. The orientation and the subsequent presentation of the immunogen is a critical quality attribute for the generation of functional antibodies specific to the antigen. These multimeric MAPs have been demonstrated to be highly immunogenic, allowing production of polyclonal and monoclonal antibodies. The majority of these antibodies react with the peptide in its monomeric form as well as its multimeric form. The antigenic determinants of the peptide that are typically recognized by these antibodies include continuous type as well as conformational type of determinants.

Several multiple antigen-presenting peptide vaccine systems have been developed and described in the art [Fujita et al., 2011; Moyer et al., 2016]. These include: (1) the addition of functional components, e.g., T-cell epitopes, cell-penetrating peptides, and lipophilic moieties; and (2) synthetic approaches using size-defined nanomaterials, e.g., self-assembling peptides, non-peptidic dendrimers, and gold nanoparticles, as antigen-displaying platforms; (3) chemical conjugation of adjuvant compounds to protein antigens [Wille-Reece et al., 2005]; (4) the incorporation of antigen or adjuvant into particulate vehicles by conjugation to the surface of nanoparticles [de Titta A, et al., 2013]; (5) the incorporation of antigen or adjuvant into particulate vehicles by the entrapment within lipid vesicles or capsules [Moon et al., 2011]; (6) the encapsulation within polymer particles [Ilyinskii, et al., 2014]; and (7) Virus Like Particles (VLPs) [Cimica et al., 2016; Donaldson et al., 2018]. Liposomes and poly(lactide-co-glycolide) (PLGA) have been described in the art as vaccine vehicles [Silva et al., 2016].

The MAP vaccines, described in the art, can carry several copies of peptide antigens on a carrier or nanoparticle and can elicit higher antibody titers than single peptide monomers and carrier protein-peptide conjugates. However, the main limitation in the MAP systems is the need for additional components, such as an adjuvant, in many cases to elicit robust immunogenicity. Therefore, subsequent research efforts have been directed towards improvement of these MAP vaccines by the incorporation of multiple functions into a single vaccine product using helper T-cell epitopes, immune-stimulant lipid moieties, or cell-penetrating peptides, etc.

Another approach to boost the immunogenicity of various protein antigens is through the use of adjuvants such as Aluminum and Calcium based compounds, saponin based compounds such as QS-21, squalene-based compounds such as MF-59, Monophosphoryl Lipid A, etc. [Reed et al., 2013]. Another approach to boost the immunogenicity of various protein antigens is through the use of mucoadhesive adjuvants such as the derivatives of polyglutamic acids (PGA). Several PGA derivatives have been described in the art as potential mucoadhesive adjuvants. For example, a PGA-based complex has been described as an efficient mucosal adjuvant system for an influenza vaccine based on the recombinant fusion protein sM2HA2, which contains the consensus matrix protein 2 (sM2) and the stalk domain of HA (HA2) [Noh et al., 2019] . The γ-PGA synthesized naturally by microbial species (e.g. Bacillus subtilis and Bacillus licheniformis) is a highly anionic polymer that is used in a variety of applications (e.g., food products, cosmetics, and medicines) and has been shown to have excellent biocompatibility and noncytotoxicity [Buescher et al., 2007, Kim et al., 2007]. It can act as a mucoadhesive delivery vehicle for recombinant protein antigens and also provide an easy and robust strategy for the incorporation of hydrophobic immunostimulatory compounds such monophosphoryl lipid A (MPL) derivatives, QS21 and intracellular stimulator of interferon genes (STING) agonist adjuvants. The current “antigen plus adjuvant” vaccine products are comprised of two key components “antigen” (drug substance) and “adjuvant” followed by a formulation process step which may contain additional inert ingredients such as “stabilizers” and “excipients” to form the drug product. In this context, it is important that the biodistribution and pharmacokinetics of the vaccine are optimized and controlled for robust immunogenicity (most potent and specific immune response towards the antigen in question), stability and safety.

There are several outstanding challenges associated with the production of optimal vaccine constructs against infectious disease and tumors [Moyer et al, 2016]. For example, several soluble protein vaccines containing mono-antigens often fail to generate a robust immunogenic response resulting in suboptimal efficacy. In the case of prophylactic vaccines, one of the key requirements to elicit a robust immunogenic response that results in an optimal efficacy is the production of a sufficient number of long-lasting antibody-producing cells and memory T and B-cell populations. For immunotherapeutic vaccines against cancer strong CD 8+ T-cell responses are required.

Antigen architecture in a vaccine construct, which defines epitope density features such as spacing, density and the rigidity/flexibility may significantly influence B cell responses, based on data from animal studies. There are several methods described in the art that demonstrate high density protein antigens, increased valency through multimerization or conjugation to polymers/carrier proteins, or multiple antigen display from nanoparticles result in the increase of B-cell triggering, antigen internalization and presentation to T-helper cells in animal models. It has been described in the art that B cells optimally recognize viruses and bacteria that typically express dense, arrayed repetitive copies of proteins at their surfaces.

It has been described in that art that peptides with specific amino acid sequences, having certain helical or β-hairpin/sheet secondary structures, can assemble themselves to form nanoparticles via non-covalent interactions such as van der Waals bonds, electrostatic interactions, hydrogen bonds or stacking interactions, etc. These nanomaterials have found applications in several fields such as tissue engineering, drug delivery, vaccine development, etc. [Hartgerink et al., 1996; Rajagopal et al., 2004].

It is also well known in the art that assemblies of polypeptides that present antigens with optimal density in defined orientations can potentially mimic the repetitiveness, geometry, size, and shape of the natural host-pathogen surface interactions [Lopez-Sagaseta et al., 2016]. Such nanoparticles offer a combined strength of multiple antigen binding sites (avidity) to provide enhanced stability and robust immunogenicity. The self-assembling properties could be leveraged for the display of various immunogens in order to mimic the repetitive display architecture of a natural microbe, e.g. a virus capsid.

Several exogenous multimerization domains that promote formation of stable multimers of soluble proteins are known in the art. Examples of such multimerization domains that can be linked to an immunogen provided by the present disclosure include: (1) the GCN4 leucine zipper [Harbury et al. 1993]; (2) the trimerization motif from the lung surfactant protein [Hoppe et al. 1994]; (3) collagen [McAlinden et al. 2003] and (4) the phage T4 fibritin foldon [Miroshnikov et al. 1998].

It has been described in the art that coiled-coil sequences in proteins which consist of heptad repeats containing two characteristic hydrophobic positions have been exploited for self-assembly for the production of nanoparticles [Miroshnikov et al., 1998; Todd et al., 2002; McAlinden et al.,2003]. The role of these buried hydrophobic residues in determining the structures of coiled coils was investigated by studying mutants of the GCN4 leucine zipper [Harbury, 1993]. Short stretches of amino acids can form structural motifs responsible for the tight parallel association and trimerization of the three identical polypeptide chains of lung surfactant protein D, which contains both collagen regions and C-type lectin domains [Hoppe 1994].

It has also been described in the art that several Self-assembling Peptidic Nanoparticles (SANPs) composed of a pentameric coiled-coil oligomerization domain derived from cartilage protein [Jung et al., 2009] and a trimeric coiled-coil oligomerization domain [Rudra et al., 2010] have been exploited to produce as multiple antigen-display platforms [Burkhard et al., 2001; Pimantel et al., 2009; Kaba et al., 2009]. The above described nanoparticles play a wide variety of physiological roles and this could be exploited more widely in the arena of vaccine design and development. It is also well known in the art that in silico molecular modeling approaches could be used to design such self-assembling nanoparticles for vaccine design [Lapelosa et al., 2009].

It is known in the art that the crystal structure of peptide epitopes in complex with the corresponding broadly neutralizing human monoclonal antibody have the potential to provide targets for structure-based vaccine design strategies aimed at identifying optimal antigenic activity [McLellan et al., 2013].

The Cholera Toxin B (CTB) subunit of Cholera Toxin protein, produced by Vibrio cholerae consists of a homopentameric structure that is approximately 55 kD (11.6 kD monomers) and binds to the GM1 ganglioside; found in lipid rafts, on the surface of intestinal epithelial cells [Baldauf et al., 2015]. When CTB is chemically or genetically conjugated to poor immunogens, it can elicit serum and secretory antibodies against the fused antigens [Harakuni et al., 2005]. CTB acts as a transmucosal carrier delivery system for induction of oral tolerance when conjugated to antigens and allergens. CTB has the ability to deliver covalently attached antigens to the mucosal cells via binding to GM1 ganglioside receptor on the surface of epithelial cells [Bergerot et al., 1997].

A number of methods are known in the art that describe the utility of natural or engineered protein nanoparticle scaffolds to add heterologous epitopes or antigens onto the ‘nanoparticle ’, to produce ‘chimeric’ nanoparticle antigens. These chimeric nanoparticles can be generated by self-assembly, or by covalent chemical conjugation of an antigen/immunogen to a nanoparticle.

Examples of chimeric nanoparticles include virus-like particles (VLPs) composed of single or multiple viral antigens, in some instances anchored in a lipid bilayer. A number of methods have been described in the art that involve usage of proteins from various microorganisms as templates for the production of such nanoparticles and for the presentation of immunogenic epitopes. Phage particles have been described in the art as an attractive antigen delivery system to design new vaccines [Prisco et al., 2012]. For example, filamentous phage fd has been identified as an antigen delivery platform for peptide vaccines for immunotherapeutic targets. Peptides displayed on the surface of filamentous bacteriophage fd were shown to induce humoral as well as cell-mediated immune responses. The immune response induced by phage-displayed peptides can be enhanced by targeting phage particles to the antigen presenting cells, utilizing a single-chain antibody fragment that binds a dendritic cell receptor. Other examples include the protein pIII of the filamentous phage f1, the Ty component from Saccharomyces cerevisiae, the surface and core antigens of the hepatitis B virus, surface or coat proteins of human parvovirus B19, Sindbis virus, and papillomavirus.

Examples of the vaccines based on the use of self-assembling VLPs that exploit the design principles described above include the licensed human papilloma virus vaccines, hepatitis B vaccine, etc. A malaria vaccine candidate RTS, was developed using the above design principles. It has been recommended for licensure by EMEA, after undergoing large scale phase 3 evaluation [Mahmoudi et al., 2017], was introduced in Ghana in April 2019. This vaccine is based on the hepatitis B surface antigen VLP platform, which includes the C-terminal (amino acids 207-395) of the Plasmodium falciparum circumsporozoite (CS) antigen along with the GSL ASO1 adjuvant, a mixture of liposomes, MPL and QS-21. The vaccine formulation with the mixture of adjuvants was demonstrated to induce humoral and cellular immune responses to the antigen.

A number of new vaccine design approaches have been described in the art, aided by the identification of human monoclonal antibodies with high specificity for critical immunogenic epitopes [Melero et al., 2016]. In conjunction with the capability of modern molecular modeling tools to obtain atomic-level structural information for protein antigens and precision engineering to produce self-assembling nanoparticles, clinical proof-of-concept for structure-based vaccine design may first be achieved for respiratory syncytial virus (RSV). These strategies address the longstanding challenges in vaccine development with respect to conformation-dependent access to neutralization-sensitive epitopes of protein immunogens which could determine the capacity to induce potent neutralizing activity [Graham et al., 2019]. Success with RSV has motivated structure-based vaccine design and stabilization of other protein antigens for use as immunogens.

Helicobacter pylori ferritin has been employed to develop a vaccine candidate which elicited broadly neutralizing H1N1 antibodies in animal studies [Kanekiyo et al. 2013]. Ferritin is a natural protein that can be found in cells from all living species. Ferritin is useful as a vaccine platform since it provides particles that can display multiple antigens on its surface, mimicking their natural organization. Hemagglutinin (HA) of influenza virus was inserted at the interface of adjacent subunits to generate eight trimeric viral spikes on the surface of ferritin nanoparticle via self-assembly [Darricarrere et al., 2018]. A candidate vaccine using this influenza-ferritin self-assembling nanoparticle vaccine entered Phase 1 clinical trials in 2019 [NIH News, 2019]. A prototype universal influenza vaccine which displays part of HA (stem region only) on the surface of a nanoparticle made of nonhuman ferritin was developed by NIH scientists. This H1N1 candidate vaccine protected animals from infection of H5N1 a different influenza subtype, indicating that the antibodies induced by the vaccine can protect against other influenza subtypes within “group 1,” which includes both H1 and H5.

Several carrier proteins have been described in the art demonstrating their use in the design, development and production of licensed conjugate vaccines: a genetically modified cross-reacting material (CRM₁₉₇) of diphtheria toxin, tetanus toxoid (T), meningococcal outer membrane protein complex (OMPC), diphtheria toxoid (D), and H. influenzae protein D (HiD). The efficacy of these conjugate vaccines has been successfully demonstrated in the form of preventing infectious diseases and controlling the spread of Haemophilus influenzae b, Streptococcus pneuomoniae, and Neisseria meningitidis [Pichichero, 2013]. Even though these carrier proteins have been effective in increasing vaccine immunogenicity, they differ in the quantity and avidity of antibody they elicit and the number saccharide antigen units in the same product.

While CRM₁₉₇ and tetanus toxoid are frequently used in commercial products, the criteria for the selection of an optimal choice for the selection of a carrier protein are unclear. Therefore, there is a need to develop a better understanding of T-cell responses induced by carrier proteins and differences among carrier proteins. Assuming that presentation of antigen attached to carrier protein peptide to T cells via uptake to MHCII is a systematic/universal process, it is likely that certain peptide fragments are better than others. This requires discovering the carrier protein epitopes presented to and recognized by helper T cells from conjugate processing in antigen-presenting cells [Avci et al., 2019]. In this context, the principles of structural vaccinology should be leveraged towards carrier protein design. Ultimately, carrier proteins should be designed to achieve optimal T-cell presentation.

The superiority of conjugate vaccines, relative to the unconjugated haptens and antigens, is derived from their ability to induce immunologic memory [Pichichero, 2013]. The mechanisms by which this immunologic memory is established involves activation of T-helper cells leading to generation of both memory B-cells and memory T-cells. There are several factors that determine the robustness of the immunologic memory that effectively results in bactericidal activity: (i) the kinetics of memory response for memory B-cell re-activation until maturation to antibody-secreting plasma cells occurred, which in turn is controlled by memory B-cell exposure to antigen and consequent production of detectable antibody (ii) the avidity of the antibodies; (iii) the level of serum antibody; (iv) the specificity of the serum antibody and (iv) persistence of serum antibody levels following primary and booster vaccinations.

Continuing additions of new vaccines to routine infant immunization schedules have raised concerns about potential interactions between vaccine components reducing desired efficacy results [Avci et al., 2019]. Consequently, there is a potential concern related to weakening of antigen-antibody interactions that may ultimately dilute the desired immunogenicity levels and vaccine efficacy. In addition, the introduction of multivalent combination conjugate vaccines has also led to some efficacy related challenges, such as interference in the immunogenicity profile [Pollabauer et al., 2009; Pichichero, 2013]. It has been postulated that there are two major mechanisms of immunologic interference: (i) antigen competition and (ii) Carrier Induced Epitope Suppression (CIES). In case of antigen interference, one of the mechanisms that has been postulated for antigen competition among combination vaccines is antigen processing or transport. On the other hand, the mechanism for CIES has been postulated to involve inhibition in the presentation of the polysaccharide antigen epitopes, on a carrier protein, due to concurrent immunization and continued use of the same protein carrier in the combination multi-valent conjugate vaccines. For example, the concern related to increasing loads of carrier protein is the potential interference with immune responses to polysaccharide components of co-administered glycoconjugate vaccines.

Pollabauer et al have concluded, based on a critical appraisal of existing evidence from multiple clinical trials of glycoconjugate vaccines, that neither the carrier protein type nor dose adequately explains observed carrier protein related interference. For example, in five clinical trials, actually enhancement in the levels of anti-polyribosylribitol phosphate Haemophilus influenzae type b antibody was observed after co-administration of monovalent meningococcal C conjugate vaccine with tetanus toxoid carrier, not interference. Therefore, empirical observations do not seem to fit well with CIES alone as an underlying mechanism of interference. Therefore, co-administration of conjugate vaccines can have positive as well as negative effects, and predictors of vaccine interactions are still not very clear due to a number of confounding factors and potential product-based variables. In light of this, proper control of the formulation with respect to the consistency of antigen presentation of the product is critical. Removal the product quality as a variable is important in order to alleviate concerns related to antigen interference as well as CIES. Since antigen competition and/or CIES may play a role in reducing vaccine efficacy; this challenge is a major consideration during the design strategy and development of each new conjugate vaccine construct for optimal immunogenicity. The possibility of vaccine interference should be an important consideration when co-administering new multicomponent and multi-valent conjugate vaccines.

The currently available strategies practiced in the vaccine design and development discussed above, however, address only the key features required for the ‘optimal drug substance’ and not necessarily an ‘optimal drug product’. However, it is important to note that for many vaccine candidates the role of adjuvants provides an additional layer of assurance for the development of an optimal vaccine construct towards the generation of a durable and robust immunogenic response.

The design features of the optimal vaccine construct, therefore, should incorporate the particle shape to mimic the microbial structure such as the antigen display architecture on the particle surface and repetition pattern (antigen density/copy number). In addition, an integrated delivery of the antigen and adjuvant as well as the stability are important, as part of a comprehensive vaccine design and development strategy [Moyer, 2016; Prasad, 2018].

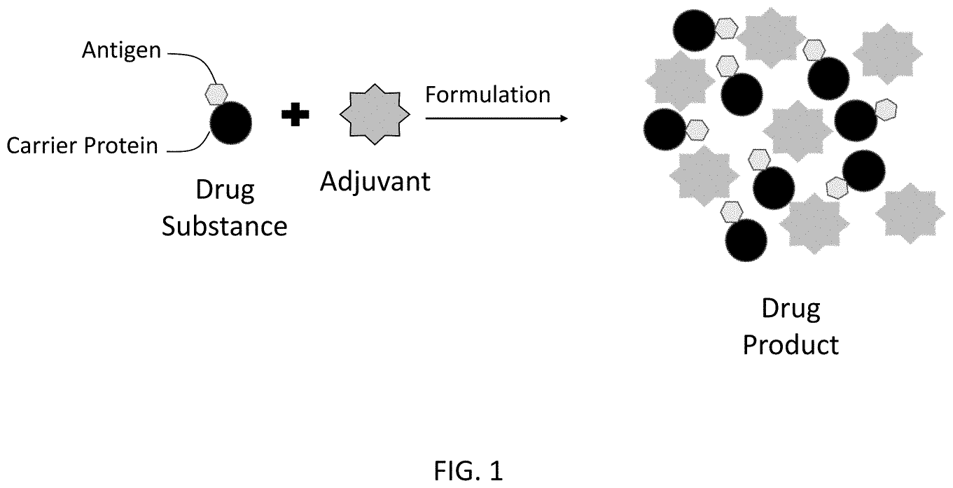

The formulation process, currently used for the manufacture of various vaccines to produce the final Drug Product (DP) with various adjuvants such as aluminum phosphate, aluminum hydroxide, calcium phosphate etc., is typically not a fully controlled process step (FIG. 1). These adjuvants have been used for several decades to enhance the immune response to vaccines. The control of the formulation process parameters is vital for manufacturing consistency since they have a direct impact on the physical, chemical, and biological properties of these adjuvants [HogenEsch 2018]. The optimization of the final construct of the vaccine could ultimately determine vaccine performance. The non-specific interactions between adjuvants and antigens, therefore, may have a direct impact on the potency of vaccines.

Vaccine products encounter various types of interfacial stress during development, manufacturing, and clinical administration [Li et al., 2019]. Protein antigens come in contact with various surfaces during various steps of formulation, with adjuvants, excipients and stabilizers. These additional interfaces can negatively impact the final vaccine drug product quality attributes. During the various processing steps of the vaccine drug substance including final formulation, additional chances for the formation of undesirable modifications and side products could arise. These undesirable modifications include formation of visible particles, subvisible particles, or soluble aggregates and/or changes in target protein concentration due to the potential adsorption of the molecule to various interfaces. Protein aggregation at interfaces is often accompanied by changes in conformation, in response to interfacial stresses such as hydrophobicity, charge, and mechanical stress. Formation of aggregates due to these non-specific interactions may bury critical immunogenic epitopes from optimal interactions leading to suboptimal immunogenicity. Therefore, it is important to minimize opportunities for aggregation throughout the product design and development cycle and to develop appropriate mitigation strategies.

The current classical formulation process, widely used in the manufacture of licensed vaccines as well as most clinical candidates, typically involves mixing of various vaccine antigens (drug substances) with a given adjuvant such as an aluminum salt, using a few control parameters such as excipients, salts, pH, adjuvant concentration, etc., to produce the final drug product. However, this control of the formulation process using a few selected process parameters results only in the partial control of the formulation process. The partial control of formulation process parameters, without addressing the key aspect of the optimal presentation of the critical immunogenic epitopes, may result in the random burial of the critical immunogenic epitopes, in the final formulated drug product, preventing their ability to interact with the antigen presenting cells (APCs) (FIG. 1).

It is, therefore, important that this key aspect of optimal antigen as well as the T-cell epitope presentation in the final formulated drug product is addressed in an adequate manner to elicit a robust immunogenic response. In addition to the optimal presentation of the critical immunogenic epitopes and T-cell epitopes, it is also important to control the antigen number (antigen density) in a consistent manner. The formulation processing, using adjuvants, without proper optimization due to incomplete understanding of the orientation and presentation critical immunogenic epitopes and T-cell epitopes may also result in the choice of an inappropriate adjuvant. In addition to the presentation, adsorption of the antigen results in the conversion of the soluble antigens to particulate form, which enhances uptake through phagocytosis by dendritic cells. The proper control of the process of adsorption during the formulation step also results in the control of key attributes of the particulates, such as molecular size. The process of adsorption to the adjuvant also results in the retention of the antigen at the injection site, allowing time for recruitment of APCs through release of cytokines and the induction of a local inflammatory reaction. The proper control of the process of adsorption during the formulation step, therefore, results also in the control of key attributes that define the retention of the antigen at the injection site.

An optimal conjugate vaccine formulation, therefore, requires that four key criteria are met in order to elicit a robust immunogenic response resulting in efficacy (i) minimization of non-specific interactions between the conjugated antigen, the T-cell epitopes of the carrier protein and the adjuvant; (ii) transport of conjugate vaccines to lymphoid tissues, (iii) trigger conjugated antigen as well as T-cell induced signals to immune cells, and (iv) control of the kinetics of conjugated antigen as well as the T-cell epitope presentation to immune cells. An integrated strategy for the delivery of the conjugated antigen, along with the T-cell epitopes, and the adjuvant in a concerted manner to the lymphoid cells helps to address the four key criteria outlined above. These integrated design features and the formulation strategy ultimately are the drivers that help shape the elicitation of an optimal and robust immunogenic response, along with T-cell help, as well as the stability of the vaccine construct.

It is the object of the current invention to address the proper control of the formulation, with respect to the consistency of antigen presentation of the product in order to alleviate concerns related to antigen interference as well as CIES. The current invention is specifically geared towards addressing the proper control of the formulation, with respect to the consistency of critical antigen epitopes presentation of the product, in order to alleviate potential concerns related to antigen interference. The current invention is also geared towards addressing the proper control of the formulation, with respect to the consistency of presentation of the critical T-cell epitopes of the carrier protein, in order to alleviate potential concerns related to CIES. The proper control of the formulation with respect to the consistency of antigen presentation as well as the T-cell epitopes of the product, designed using rationally engineered Carrier Proteins (reCaP), is the primary objective of the current invention, to remove the product quality as a variable and alleviate potential concerns related to antigen interference as well as CIES.

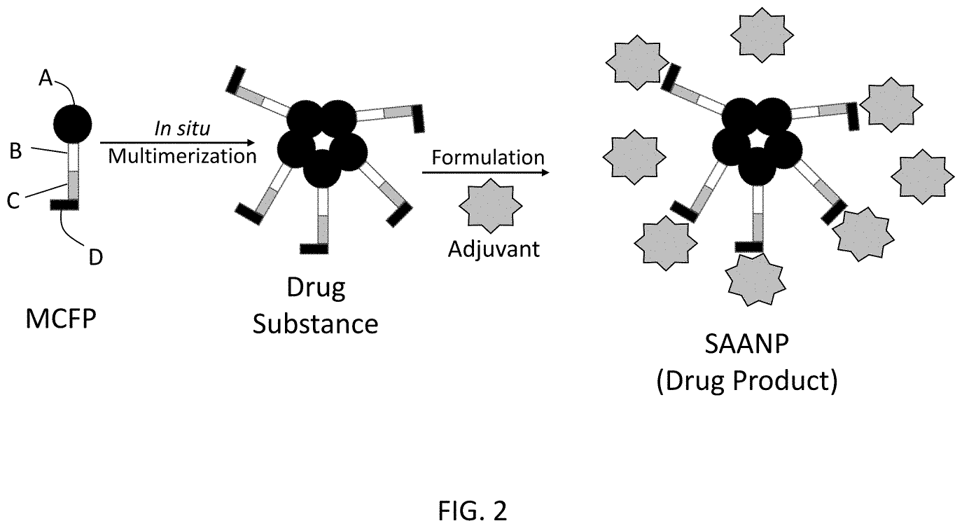

It is the object of the current invention to incorporate the above described design features and address the four key criteria, listed earlier, by production of integrated Multiple Antigen displayed Adjuvant Systems (iMAAS), comprising antigens and rationally engineered carrier proteins (reCaP), for immunogenicity enhancement as well as enhanced stability (FIG. 2).

By genetically fusing two or more polypeptide and protein domains together, fusion proteins may elicit many specific biological functions derived from each of their component moieties. In addition to their broad applications in biological research such as protein purification and imaging, recombinant fusion proteins have also become an important category of biopharmaceuticals [Chen et al., 2013]. In addition to the fundamental role in stitching functional domains together, linkers may offer many additional advantages for the production of fusion proteins, such as enhancement of biological activity, higher expression yield, and achieving optimal pharmacokinetic profiles. Several fusion proteins drugs including tumor necrosis factor/Fc-IgG1, Interleukin-2/diphtheria toxin, Cytotoxic T-Lymphocyte Antigen-4/Fc-IgG1, Leukocyte function antigen-3/Fc-IgG1, Interleukin-1 Receptor extracellular domain/Fc-IgG1, and thrombopoietin/Fc-IgG1) have been licensed for human use by FDA.

Therefore, in addition to the protein carriers currently used in licensed conjugate vaccines as well as many clinical candidates, there is a need for new carrier proteins. This need is driven by several considerations: (i) to address antigen interference and/or CIES, leading to reduction of the specific immune response; (ii) explore the dual role of proteins as carrier and protective antigen; and (iii) optimizing antigen-antibody interactions. Selection criteria for new protein carriers are based on several aspects including safety, manufacturability, stability, reactivity toward conjugation, and preclinical evidence of immunogenicity of corresponding glycoconjugates [Micoli et al., 2018].

Integrated Multiple Antigen Displayed Adjuvant Systems (iMAAS)

The integrated Multiple Antigen displayed Adjuvant Systems (iMAAS) approach, comprising reCaPs, is geared towards producing chimeric fusion protein vaccines that are more immunogenic and/or more stable compared to the corresponding standalone soluble monomeric vaccine antigens.

A key aspect of this iMAAS is to produce Chimeric Fusion Proteins (CFPs) by incorporating Dual Function Peptides (DFP) which aid in the purification process as well as participate in the selective binding to various adjuvants. By functioning as purification aids, the DFPs can simplify the purification process, thereby reducing the number of unit operations and cost of production. This is particularly useful to produce vaccines that are rapidly deployable during pandemic disease situations. The selective non-covalent affinity binding capability of the DFPs with the adjuvant can help in multimerization of the antigens in addition to minimizing the non-specific interactions between the antigen and the adjuvant (FIG. 2). The selective binding capability of the DFPs can help in the optimal display of the critical immunogenic epitopes by minimizing the non-specific interactions between the vaccine antigen and the adjuvant.

In some aspects, the present invention provides a strategy by which carrier proteins could be genetically engineered to produce Multifunctional Chimeric Fusion Proteins (MCFPs) comprising: (a) linker peptides (b) polypeptide antigens (c) Dual Function Peptides (DFP) and (d) carrier proteins. The purpose of engineered DFP is to function both as a purification aid as well as having the non-covalent affinity to selectively bind to adjuvants (FIG. 2). In the first step, the genetically engineered MCFPs are produced using recombinant protein expression. Escherichia coli is one of the organisms of choice for the production of MCFPs. Its use as a cell factory is well-established and it has become the most popular protein expression platform. A number of molecular tools and protocols have been described in the art for the high-level production of heterologous proteins, such as a vast catalog of expression plasmids, a great number of engineered strains and many cultivation strategies.

During the purification of a MCFP, it is invaluable to have an aid or peptide tag to (i) detect it along the expression and purification scheme, (ii) attain maximal solubility, and (iii) easily purify it from the E. coli cellular milieu. The expression of a stretch of amino acids (peptide tag, in the form of DFP) in tandem with the desired reCaP to form a CFP may allow these three objectives to be achieved in a straightforward manner.

A number of vectors have been described in the art that allow positioning of the DFP at either the N-terminal or at the C-terminal end. The C-terminus option is advantageous such that a signal peptide can be placed at the N-terminal end for secretion or periplasmic deposition of the recombinant protein. Common examples of small peptide purification tags are the poly-Arg-, FLAG-, poly-His-, c-Myc-, S-, and StrepTag II-tags [Rosano et al., 2014]. Purification tags, such as the ones listed above, allow for one-step affinity purification, as resins that tightly and specifically bind the tags are available. For example, His-tagged proteins can be recovered by immobilized metal ion affinity chromatography (IMAC).

In the next step, the MCFP (drug substance, DS) is formulated with an adjuvant and optionally with other excipients and stabilizers (FIG. 2).

During the formulation process of a MCFP, it is invaluable to have an aid or peptide tag to (i) selectively bind to the adjuvant and (ii) prevent non-specific binding of the critical immunogenic epitopes of the MCFP to the adjuvant. The recombinant expression of a stretch of amino acids (e.g. a peptide tag, in the form of DFP) in tandem with the desired reCaP to form a CFP may allow these twin objectives.

The two key design features of DFP are (i) acting as a purification aid and (i) non-covalent affinity binding to various adjuvants by ionic charge or hydrophobic affinity or van der Waals forces or coiled-coil binding. For example, positively charged histidine affinity tags can function as purification tags as well bind to negatively charged mucoadhesive adjuvants such as polyglutamic acid (PGA) derivatives. It is important to choose a drug product buffer matrix as well as the pH (range 5.5-7.5) that maximizes the binding between DFP and the adjuvant. The optimal range for MCFP and the adjuvant binding needs to be determined for each specific peptide antigen vaccine construct comprising the MCFP.

The examples illustrated by the current invention by which these MCFPs, comprising Rationally Engineered Carrier Proteins (reCAPs), could be produced for immunogenicity and stability enhancement, directed towards producing a robust immunogenic response.

The current invention also provides an illustration by which the efficiency of a carrier protein, using CTB as an example, could be further augmented by designing MCFPs with the incorporation of various peptide antigens, in conjunction with DFPs, to generate SAANPs with the ultimate goal of producing optimal vaccine constructs (e.g., SEQ ID NOs: 6, 8, 21, 23, 25, 31, 35, 40, 43, 46, 50, 54)

The current invention also provides an illustration by which the efficiency of a carrier protein, using CTB as an example, could be further augmented by designing MCFPs with the incorporation of various peptide antigens, in conjunction with DFPs, to generate SAANPs with the ultimate goal of producing optimal vaccine constructs (FIG. 2).

The current invention also provides an illustration by which the efficiency of a carrier protein, using CTB as an example, could be further augmented by the incorporation heterologous peptide antigen epitopes derived from various protein antigens (e.g., SEQ ID Nos: 31, 35, 40, 43, 46, 50 and 54).

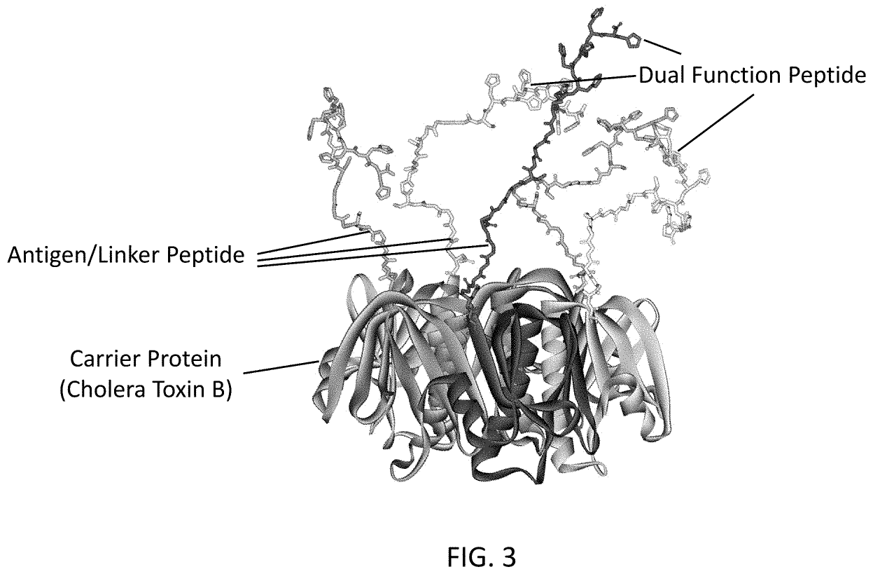

The inventor has surprisingly found that chimeric fusion proteins with peptides linked at the C-terminus of CTB have favorable expression as well as solubility characteristics compared to the chimeric fusion proteins produced with peptides linked at the N-terminus of CTB. The protein model of the CTB displaying a linker peptide (GGGSGGGSGGGS) and Histidine6Tag at the C-terminus is shown in FIG. 3 (SEQ ID NO: 25). Optionally, the linker peptide could be replaced or added with a heterologous T-cell epitope.

It is also the object of the current invention to incorporate the above described design features and the four key criteria, listed earlier, to produce integrated Multiple Conjugated Antigen displayed Adjuvant Systems (iMCAAS), comprising conjugated antigens and rationally engineered carrier proteins (reCaP), for immunogenicity enhancement as well as enhanced stability. Specifically, the current invention is also directed towards incorporating these dual design features of both the antigen display as well as reCaP display, to produce Self Assembling Adjuvanted Nanoparticles (SAANPs). More specifically, the current invention describes methods and examples that provide multiple antigen peptide-linked Carrier Protein Displayed Adjuvant (CAPDAdjuvant) systems for immunogenicity and stability enhancement. It is also the object of this invention to produce nanoparticles that incorporate the particle shape to mimic the microbial structure such as the multiple antigen display architecture on the particle surface and repetition pattern (antigen density/copy number). In addition, these adjuvanted nanoparticle systems incorporate the design features for the integrated delivery of the conjugated antigen, comprising reCaP and adjuvant as well as the stability, as part of a comprehensive vaccine design and development strategy.

The iMCAAS approach, comprising reCaPs is geared towards producing conjugate vaccines more immunogenic and/or more stable compared to the standalone “conjugate vaccine” antigens.

The following examples also illustrate the method by which reCAPs as well as the CAPDAdjuvants, comprising reCaPs, could be produced for immunogenicity and stability enhancement, to address one or more challenges associated with vaccine interference and/or CIES, by producing a robust robust immunogenic response.

The following examples also illustrate the method by which these reCAPs as well as the CAPDAdjuvants, comprising reCaPs, could be produced for immunogenicity and stability enhancement, to address one or more challenges associated with vaccine interference and/or CIES, by producing a robust robust immunogenic response.

Cross-reacting material 197 (CRM₁₉₇), a single amino acid mutant of diphtheria toxoid, is a commonly used carrier protein in commercially licensed polysaccharide protein conjugate vaccines [Madore et al., 1987; Pichichero, 2013, Prasad 2018]. CRM₁₉₇carrier protein contains two T-cell epitopes of the CRM₁₉₇(amino acid residues 306-334 and 357-383) [Bixler, et al., 1989; Bixler et al., 1998], with the following amino acid sequences shown in SEQ ID NOs: 26 and 28.

A significant point of structural importance, of CRM₁₉₇ carrier protein, is the lack of lysine residues in the above described T-cell epitopes (e.g., SEQ ID Nos: 26-28). For the vast majority of the licensed polysaccharide antigen-based conjugate vaccines, as well as the clinical candidates, the conjugation of the protein to the antigen occurs via the modification of the lysine residues in CRM₁₉₇. Therefore, it is important to note that high levels of lysine modification by the activated saccharide antigens, involving CRM₁₉₇as carrier protein in the conjugation, is less likely to directly interfere with the T-cell functional aspects of the carrier protein. The lack of lysine residues, in the T-cell epitopes, presents itself as a major advantage to consider CRM₁₉₇ as the carrier protein of choice towards the design and development of conjugate vaccines [Prasad, 2018].

The current invention also provides an illustration by which the efficiency of a carrier protein, using CTB as an example, could be further augmented by the incorporation of the two heterologous T-cell epitopes derived from the protein sequence CRM₁₉₇. In this example, two T-cell epitopes of CRM₁₉₇ , one polypeptide at the N-terminus and the second polypeptide at the C-terminus. The DNA sequence for the above described hybrid carrier protein containing the primary sequence of the CTB carrier protein, flanked by the two T-cell epitopes of CRM₁₉₇is provided in SEQ ID NO: 30.

The first T-cell epitope from the CRM₁₉₇ protein sequence (region 1, amino acid residues 306-334) was fused to the N-terminus of the CTB protein, whereas the CRM₁₉₇ protein sequence (region-2, amino acid residues 357-383) was fused to the C-terminus of the CTB carrier protein. The final protein sequence of the hybrid carrier protein containing primary sequence of the CTB carrier protein, flanked by the two T-cell epitopes of CRM₁₉₇ is shown in SEQ ID NO: 31.

As illustrated by the above example using CTB, it is the objective of the current invention to optionally take advantage of the T-cell epitopes, that contain no lysine residues, derived heterogeneously and insert these polypeptides in other carrier proteins and produce CAPDAdjuvants comprising these reCaPs. One of the objectives of the current invention is the optional insertion of heterologously derived T-cell epitopes in various carrier proteins for the purpose of production of conjugate vaccines and augment the final T-cell mediated response of the resultant hybrid carrier proteins. The reCaPs could be independently used to produce conjugate vaccines or the CAPDAdjuvants that are more potent immunologically (SEQ ID NOs: 31 and 35).

Another objective of the present invention is to identify an optimal adjuvant formulation compatible with oral/sublingual/buccal immunization routes to elicit a robust mucosal immune response.

As described above, a primary goal of the iMAAS approach incorporating DFPs is to produce an easily purified, rapidly deployable vaccine for use in pandemic situations. Several novel infectious diseases have emerged over the past decades. SARS-CoV emerged as a pandemic in China in 2002 and spread to five continents through air travel routes, infecting more than 8,000 people and causing 774 deaths. In 2012, MERS-CoV emerged in the Arabian Peninsula, where it remains a major public health concern, and was exported to 27 countries, infecting a total of 2,494 individuals and claiming 858 lives. A previously unknown coronavirus, named SARS-CoV-2, was discovered in Dec. 2019 in Wuhan, China. The emergence of SARS-CoV-2, as a pandemic pathogen, has resulted in >8,014,550 infections and >436,300 deaths, as of 15 Jun. 2020. Due to its facile and rapid production, the iMAAS platform would be well suited to address these novel viral diseases.

SARS-CoV-2 binds with high affinity to human ACE2 and uses it as an entry receptor to invade target cells. The Coronavirus spike (S) glycoproteins, of SARS-CoV-2, promote entry into cells and are the main target for vaccine development. Coronavirus spike (S) glycoproteins promote entry into cells and are the main target of antibodies. It has been reported that SARS-CoV-2 S uses ACE2 to enter cells and that the receptor-binding domains (RBD) of SARS-CoV-2 S and SARS-CoV S bind with similar affinities to human ACE2, correlating with the efficient spread of SARS-CoV-2 among humans [Walls et al., 2020]. Walls et al., determined cryo-Electron Microscope structures of the SARS-CoV-2 S ectodomain trimer. The SARS CoV S murine polyclonal antibodies potently inhibited SARS-CoV-2 S mediated entry into cells, indicating that cross-neutralizing antibodies targeting conserved S epitopes can be elicited upon vaccination.

The pentameric carrier protein Cholera Toxin B (CTB) could be genetically engineered to produce MCFPs comprising: (a) linker peptides, optionally fused to heterologous T-cell epitopes (b) polypeptide antigens from the RBD of the SARS Cov-2 spike proteins and (c) DFP.

The primary goal of the rational design of MCFP, comprising a DFP, in conjunction with a linker peptide, the target antigen polypeptide epitope and the carrier protein is to have a simpler, cost-effective and rapidly deployable purification process as well as a better controlled formulation process for pandemic pathogens such as SARS CoV, SARS CoV-2, MERS, etc.

One example by which the efficiency of a carrier protein, using CTB as an example, could be further augmented by designing MCFPs with the incorporation of the peptide antigens from RBD of the spike protein S at the C-terminus, to generate SAANPs with the ultimate goal of producing optimal vaccine constructs to target COVID-19 pandemic disease (SEQ ID NOs: 36-54).

The current invention provides a specific strategy by which the carrier protein Cholera Toxin B (CTB) could be genetically engineered to produce MCFPs comprising: (a) linker peptides (b) polypeptide antigens from the Receptor Binding Domain (RBD) of the SARS Cov-2 spike proteins and (c) DFP. Examples of the SARS CoV-2 spike protein RBD polypeptide includes a 31-amino acid antigen fragment shown in SEQ ID NO: 36.

In contrast to the classical formulation approaches described above, it is also the objective of the invention to design novel ‘integrated Multiple Conjugated Antigen displayed Adjuvant Systems’ [iMCAAS], comprising Rationally Engineered Carrier Proteins, based on ‘Self Assembling Adjuvanted Nanoparticles’ [SAANPs]. These nanoparticles are designed and produced in a manner comprising assemblies of peptides that present multiple copies of conjugated antigens, comprising reCaP and their critical immunogenic epitopes as well as T cell epitopes in ordered arrays displayed on the adjuvants. Such nanoparticles contain defined orientations of the conjugated immunogens that can potentially mimic the critical immunogenic epitopes as well as T cell epitopes with repetitiveness, geometry, particle/molecular size and surface shape of the natural host-pathogen antigen-antibody interactions. It is an additional objective of the invention to design adjuvanted nanoparticles which provide an integrated strength of (a) optimal immunogen density due to multiple binding sites (avidity); (b) optimal display (orientation) of critical immunogenic epitopes as well as T cell epitopes; (c) reduce non-specific interactions between the critical immunogenic epitopes as well as T cell epitopes and the adjuvant; (d) enhanced stability in order to provide improved conjugated antigen shelf-life (storage) and robust immunogenicity.

The ultimate objective of the SAANPs is the proper orientation and presentation of immunogens, as well as the T cell epitopes of the carrier proteins, having high density (high copy number of conjugated antigens) to support multiple binding events to occur simultaneously between the nanoparticle and the host cell B Cell Receptors (BCRs). These multiple binding events, generated by the adjuvanted nanoparticles, eventually provide stronger antigen-antibody interactions compared to the low affinity interactions provided by the monovalent binding generated by standalone conjugated antigen vaccine immunogens.

It is, therefore, another objective of the current invention to optionally take advantage of the T-cell epitopes, that contain no lysine residues, derived heterogeneously and insert these polypeptides in other carrier proteins and produce Rationally Engineered Carrier Proteins (reCaPs) and produce CAPDAdjuvants comprising these reCaPs. One of the objectives of the current invention is the optional insertion of heterogeneously derived T-cell epitopes in various carrier proteins for the purpose of production of conjugate vaccines and augment the final T-cell mediated response of the resultant hybrid carrier proteins. The reCaPs could be independently used to produce conjugate vaccines or the CAPDAdjuvants that are more potent immunologically. The current invention, therefore, provides a strategy by which a number of carrier proteins suitable for the production of conjugate vaccines, supplemented with additional heterogeneous T-cell epitopes derived from other carrier proteins, such as CRM₁₉₇, which do not contain lysine residues, derived from other proteins. The supplementation of additional heterogeneous T-cell epitopes, without lysine residues, allows the carrier protein to use the lysine groups, within the protein for the primary purpose of conjugation to the antigen and derive supplemental boost of T-cell help from the heterogeneously introduced polypeptide regions.

In some embodiments, the present disclosure relates to the design and development of Rationally Engineered Carrier Proteins (reCaPs) geared towards producing Multifunctional Chimeric recombinant Fusion Proteins (MCFPs) useful as vaccine candidates. In some embodiments, the present invention also relates to conjugate vaccine technologies related to design and development of novel reCaPs for Immunogenicity Enhancement and/or stability enhancement and as vaccine candidates. In some embodiments, the invention also relates to the design and development of reCaPs geared towards producing MCFPs useful as vaccine candidates. In some additional embodiments, the present invention also relates to the recombinantly expressed Self-Assembling Adjuvanted Nanoparticles (SAANPs), comprising reCaPs fused with various polypeptide and protein antigens, useful as vaccine candidates. In some additional embodiments, the invention also relates to the design and development of reCaPs, with no lysine residues in the T cell epitopes, present in the carrier proteins, in order to prevent conjugation of the target antigen to the T-cell epitope regions. In some specific embodiments, the invention also relates to the design and development of reCaPs, with fewer lysine residues, geared towards producing conjugate vaccines with minimal cross-linking. In some additional embodiments, the present invention also relates to the production of Self-Assembling Adjuvanted Nanoparticles (SAANPs), comprising reCaPs conjugated with various antigens. More specifically, the present invention relates to the design and production of SAANPs, comprising MCFPs as well as carrier proteins conjugated with various antigens, to minimize non-specific interactions between the T-cell epitopes of the carrier proteins as well as the critical immunogenic epitopes of the conjugated antigens and the adjuvants.

The key components of the MCFPs are (i) genetically engineered carrier proteins; (ii) polypeptide antigens; (iii) linker peptides, optionally fused to heterologous T-cell epitopes; (iv) Dual Function Peptides (DFP) which can act as a purification aids as well having the non-covalent affinity to bind to an adjuvant (FIGS. 2 and 3).

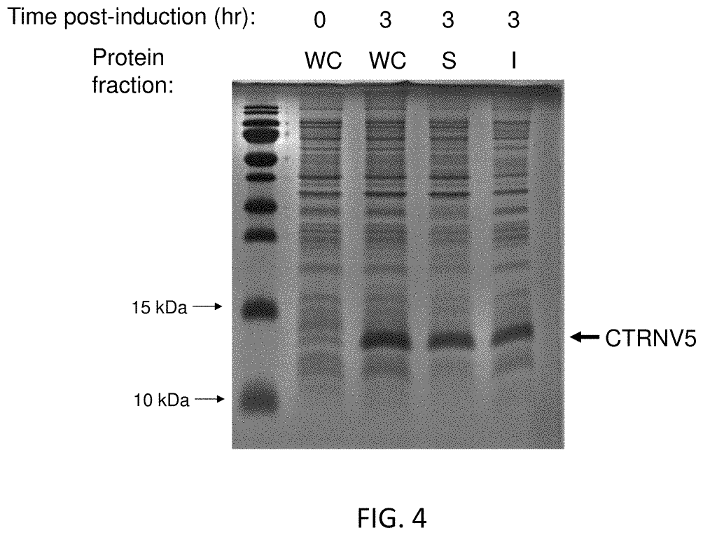

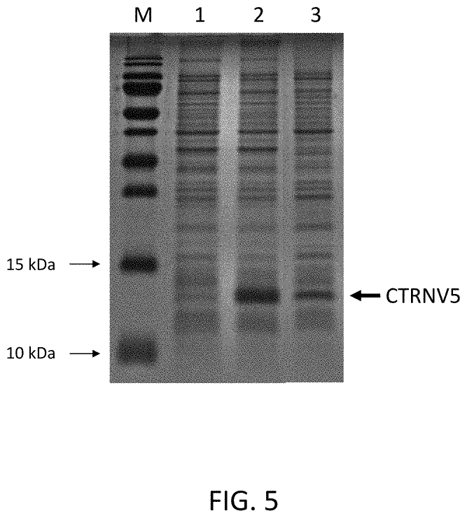

Initial efforts to express the cholera toxin subunit B (CTB) carrier protein were focused on cytoplasmic expression of a fusion protein with purification tag and linker sequences on the N-terminus (SEQ ID NOs: 6 and 8). It was found that either the protein was not detectably expressed (CTRNV1; SEQ ID NO: 6) or was expressed but completely insoluble (CTRNV2; SEQ ID NO: 8). In an attempt to improve protein solubility, signal sequences to target the recombinant protein to the E. coli periplasm were added on to the N-terminal end of the protein. These included the signal sequences from OmpA (SEQ ID NO; 13), specifically the OmpA K2V variant (SEQ ID NO: 15; Slos et al., 1994), PelB (SEQ ID NO: 17), and MalE (SEQ ID NO: 19). Linker sequences and purification tags were moved to the C-terminus. While high-level expression of the recombinant CTB protein was observed with each of these constructs, SDS-PAGE analysis of whole cell protein revealed that a substantial portion of the CTRNV3 (OmpA-CTB-(GGGS)3-His6; SEQ ID NO: 21) and CTRNV4 (Pe1B-CTB-(GGGS)3-His6; SEQ ID NO: 23) fusion proteins remained unprocessed of their respective signal sequences. Conversely, the CTRNV5 (MalE-CTB-(GGGS) 3-His6; SEQ ID NO: 25) protein was fully processed of its signal sequence, and approximately half of the protein was present in the soluble protein fraction (FIG. 4). To confirm that the processed CTRNV5 protein was indeed localized to the periplasm, a cold osmotic shock procedure was performed on cells expressing CTRNV5. SDS-PAGE analysis of the purified periplasmic protein fraction confirmed the presence of CTRNV5 in this cellular compartment (FIG. 5).

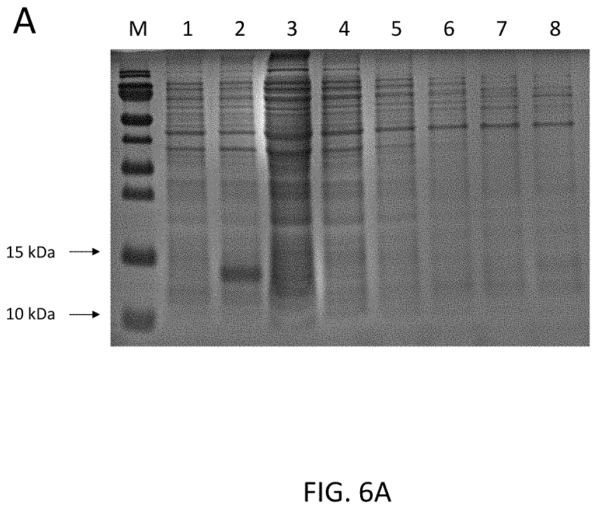

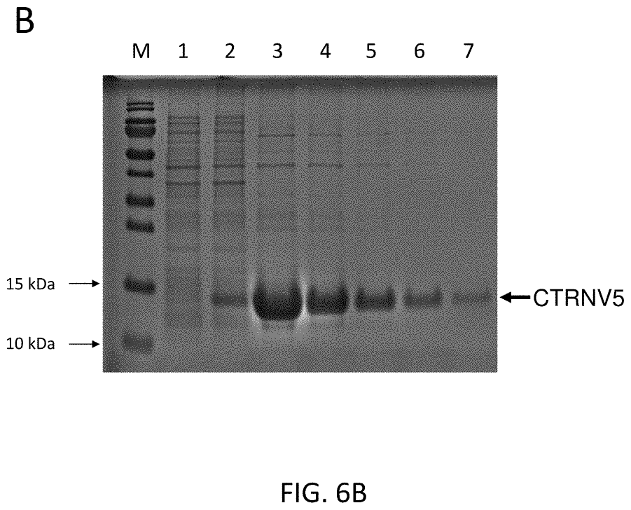

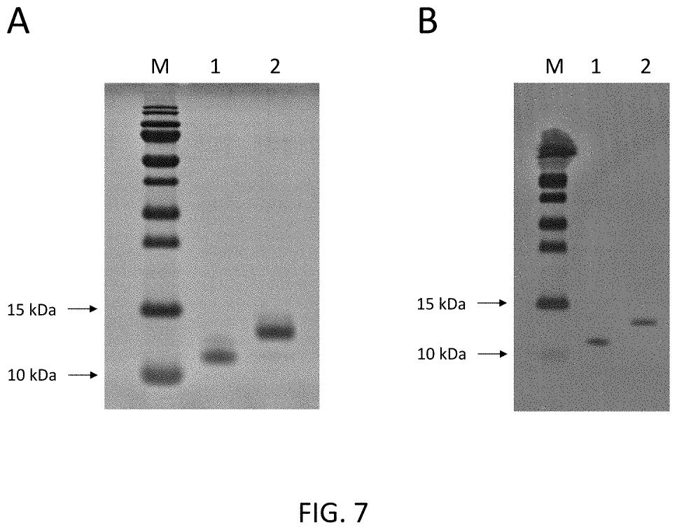

For protein purification purposes, expression of CTRNV5 was scaled-up. Immobilized metal-affinity chromatography (IMAC) was performed in batch mode on the cell lysate (FIGS. 6A and 6B). As expected, improved purity was achieved as the IMAC resin was washed with buffers with increasing concentrations of imidazole. The CTRNV5 protein was found in the elution fractions which contained 1 M imidazole. These fractions were pooled and further analyzed. To confirm the identity of the CTRNV5 purified protein, Western blot hybridization was performed with an anti-CTB monoclonal antibody, and a commercially available CTB protein was used as a positive control (FIG. 7). The antibody bound the CTRNV5 protein, consistent with the notion that CTRNV5 contains the mature CTB sequence.

The five B subunits of the CTB, which bind predominantly to GM1 ganglioside [Gal(β1-3)GalNAc(β1-4{NeuAc(α2-3}Gal(β1-4)Glc(β1 -1) ceramide] receptors found on the surface of mammalian cells, are widely thought of as delivery vehicles for various antigens. The CTB subunits possess the capacity to trigger the selective apoptosis of CD8+ T cells, as well as to alter CD4+ T-cell differentiation, activate B cells, and modulate antigen processing and presentation by macrophages [Verweij et al., 1998]. These potent immunological properties have led to testing of the B subunits as adjuvants for stimulating mucosal and systemic responses to coadministered antigens.

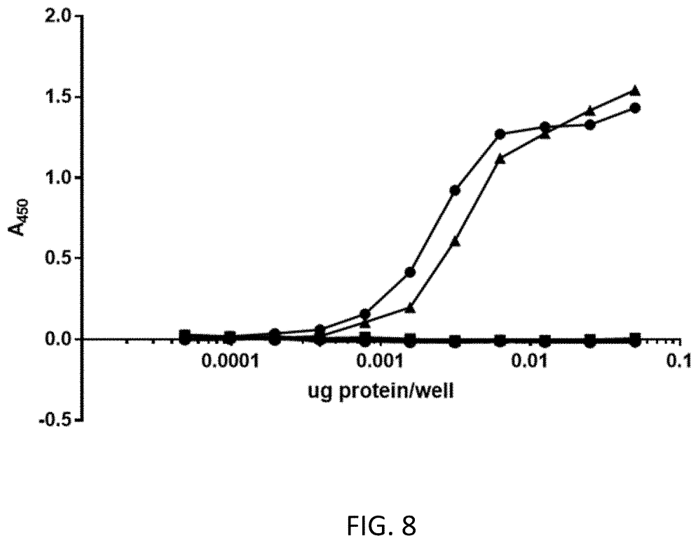

In order to demonstrate that the CTRNV5 protein is a pentamer, Enzyme-Linked Immunosorbent Assays (ELISA) were performed with the target of CTB binding, the GM1 ganglioside (FIG. 8). It has been shown previously that monomeric CTB has a low affinity for GM1 ganglioside when compared to the intact CTB pentamer (Sanchez et al., 2008). Both CTRNV5 and the positive control, commercially available CTB, were shown to bind to the plates in a GM1 ganglioside-dependent manner by ELISA; minimal binding occurred when the wells of the plate were not pre-coated with GM1 ganglioside.

The fusion protein CTRNV5, comprising CTB fusion protein (SEQ ID NOs: 24 and 25) was shown to bind with GM1 demonstrating that MCFPs produced using the current invention retain the pentameric structure. The retention of the pentameric structure is important for the functional activity and immunological properties of the MCFPs produced using the current invention to be effective as adjuvanted nanoparticles.

In some aspects, the present invention provides a strategy by which carrier proteins such as Cholera Toxin B (CTB) could be genetically engineered to produce MCFPs comprising; (a) linker peptides (b) polypeptide antigens and (c) DFPs.

In some aspects, the present invention provides a strategy by which carrier proteins such as Cholera Toxin B (CTB) could be genetically engineered, preferentially at the C-terminus, to produce MCFPs comprising; (a) linker peptides (b) polypeptide antigens and (c) DFPs.

In some embodiments, the Rationally Engineered Carrier Proteins (reCaP) contain the T cell epitopes obtained from the protein sequence of CRM197, such as amino acid residues: and 357-383 shown as follows:

T cell epitope region-1 (amino acid residues 306-334) (SEQ ID NO: 26).

T cell epitope region-2 (amino acid residues 357-383) (SEQ ID NO: 28).

In some aspects, the present invention provides a strategy by which carrier proteins such as CTB could be genetically engineered to produce MCFPs comprising; (a) linker peptides (b) polypeptide antigens derived from SARS CoV-2 and (c) DFPs.

In some specific embodiments, the current invention provides an illustration by which the efficiency of a carrier protein, using CTB as an example, could be further augmented by the incorporation heterologous peptide antigen epitopes derived from various protein antigens. The inventor has surprisingly found that chimeric fusion proteins with peptides linked at the C-terminus of CTB have favorable expression as well as solubility characteristics compared to the chimeric fusion proteins produced with peptides linked at the N-terminus of CTB.

In some specific embodiments, due to a surprising finding, the present invention provides a strategy by which carrier proteins such as Cholera Toxin B (CTB) could be genetically engineered preferentially at the C-terminus to produce MCFPs comprising ; (a) linker peptides (b) polypeptide antigens derived from SARS CoV-2 Spike Protein S and (c) DFPs.

In some specific embodiments, due to a surprising finding, the present invention provides a strategy by which carrier proteins such as Cholera Toxin B (CTB) could be genetically engineered preferentially at the C-terminus to produce MCFPs comprising ; (a) linker peptides (b) polypeptide antigens derived from the ACE2 receptor binding domains of SARS CoV-2 and (c) DFPs.

In some specific embodiments, due to a surprising finding, the present invention provides a strategy by which carrier proteins such as Cholera Toxin B (CTB) could be genetically engineered preferentially at the C-terminus to produce MCFPs comprising ; (a) linker peptides (b) polypeptide antigens derived from the ACE2 receptor binding domains of SARS CoV-2 and (c) DFPs.

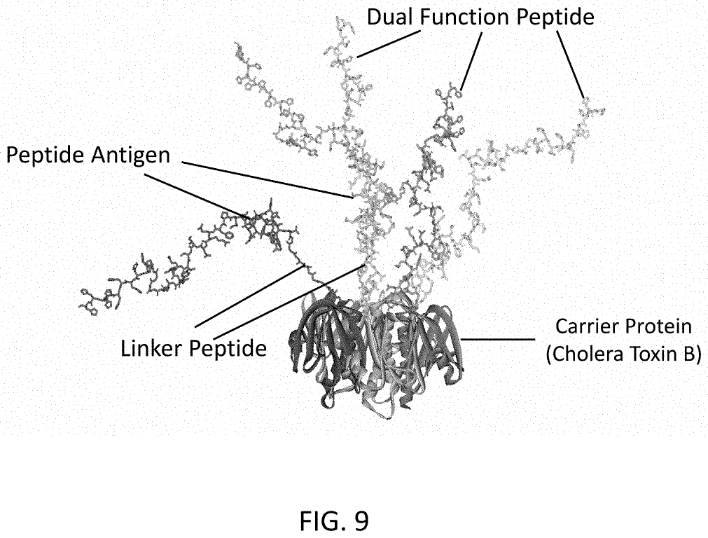

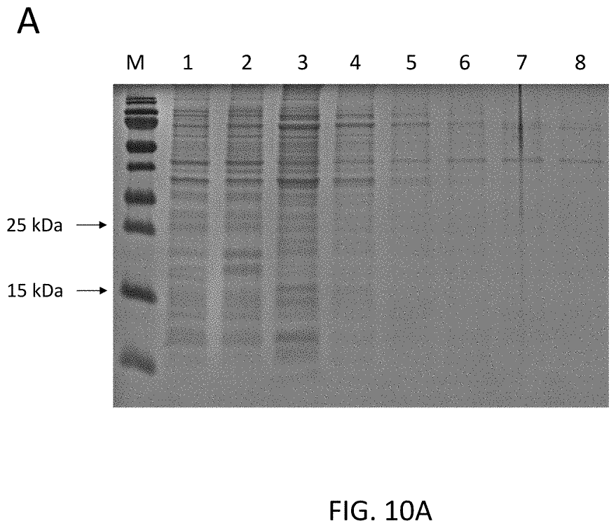

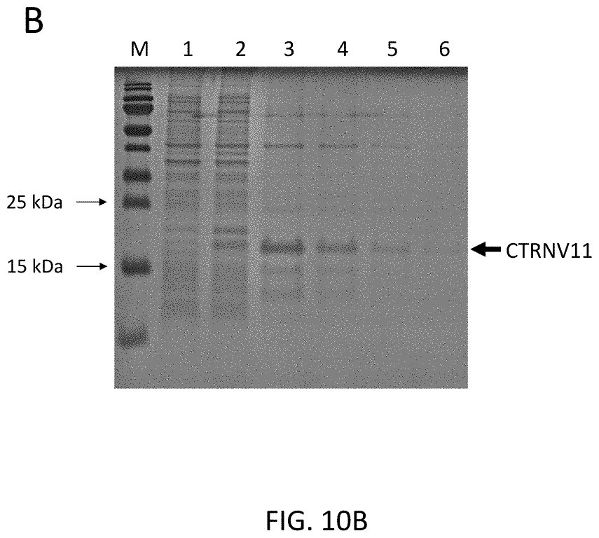

To test whether additional peptide antigens could be incorporated into the CTRNV5 carrier protein platform, an epitope from the receptor binding domain of the SARS-CoV-2 spike protein, RBD 1 (SEQ ID NO: 36), was placed on the N-terminal end of CTB (CTRNV9; SEQ ID NO: 40) or the C-terminal end of CTB (CTRNV10; SEQ ID NO: 43). The structure of this recombinant protein, CTRNV10 (SEQ ID NO: 43), is shown as a ribbon (CTB) and stick (RBD 1, linker sequences, His10-tag) diagram (FIG. 9). Small-scale expression revealed that, while the CTRNV9 protein was entirely associated with the insoluble fraction, a small portion of the CTRNV10 protein remained associated with the soluble protein fraction. Based on this observation, namely, that addition of heterologous sequences to CTB are better tolerated in terms of solubility when made on the C-terminal end, further optimizations were made. To promote folding of the CTB and RBD1 portions as discrete domains, a longer peptide linker, 3 repeats of the amino acid quadruplet GGGS, was included between CTB and RBD1. A hexahistidine tag preceded by a quadruplet GGGS linker was also included at the C-terminus. SDS-PAGE analysis revealed that the protein was expressed at high levels. While some of the recombinant protein was unprocessed of its MalE signal sequence, all CTRNV11 protein (SEQ ID NO: 46) associated with the soluble protein fraction was processed. CTRNV11 could be purified by IMAC performed in batch mode, where the recombinant protein was present in elution fractions containing 1 M imidazole (FIGS. 10A and 10B).

In some specific embodiments, the DFPs are ionically charged residues that can bind non-covalently to various adjuvants.

In some specific embodiments, the DFPs are positively charged residues that can bind non-covalently to various adjuvants.

In some specific embodiments, the DFPs are positively charged residues that can bind non-covalently to various mucoadhesive adjuvants, such as the derivatives of polyglutamic acid (PGA).

In some specific embodiments, the DFPs are derived from polyhistidine tags that can bind non-covalently to various adjuvants.

In some specific embodiments, the DFPs are derived from polyhistidine tags comprised of six to ten contiguous histidine residues that can bind non-covalently to various adjuvants.

In some specific embodiments, the DFPs are derived from polyhistidine tags that can bind non-covalently to various mucoadhesive adjuvants, such as the derivatives of polyglutamic acid (PGA).

In some specific embodiments, the DFPs are derived from polyhistidine tags comprised of six to ten contiguous histidine residues that can bind non-covalently to various mucoadhesive adjuvants, such as the derivatives of polyglutamic acid (PGA).

The present invention is directed to the methods for design of novel multi-layered iMCAAS for immunogenicity and stability enhancement, applicable for the development of optimal nanoparticle vaccine constructs. The classical approach of mixing of the conjugated antigen(s) with adjuvant(s) during the formulation (FIG. 1). Several advances have been made towards addressing the proper orientation and density of the conjugated antigen along with presentation and shape of the immunogenic constructs (drug substance portion), as exemplified by Self-Assembling Nanoparticles (SANPs). However, the key challenge of the protection of the critical immunogenic epitopes as well as T cell epitopes from unwanted or non-specific interactions that occur during the mixing with the adjuvant using the SANPs remains unaddressed, since this method still involves using the partially controlled classical formulation process approach. In one embodiment, the present invention is directed to the methods for design of novel multi-layered iMCAAS for immunogenicity and stability enhancement, applicable for the development of optimal nanoparticle vaccine constructs. In other embodiment, these novel Multiple CAPDAdjuvant systems are constructed more specifically using three inventive steps comprising (i) covalently linking specific short peptides, without lysine residues, to the carrier proteins for the purpose of formation of multimeric arrays ; (ii) chemical conjugation of the peptide-linked carrier protein to the antigen of interest; and (iii) the final formulation process step, which directs the multiple antigen arrays of the conjugated antigen on the adjuvant in a well ordered and controlled manner to produce CAPDAdjuvants, to reduce non-specific interactions.

In another embodiment, the invention provides iMCAAS to be used as efficient conjugate vaccines comprising ‘integrated functionalized carrier protein nanoparticles containing both an conjugated antigen and an adjuvant’, and a method of vaccinating humans or non-human animals using such functionalized conjugated antigen, comprising rationally engineered carrier proteins, nanoparticles containing adjuvants. The invention also provides processes for making conjugated antigen nanoparticles comprising functionalized carrier protein nanoparticles.

The present invention is specifically directed to address the key challenge of the proper orientation and the preservation, protection and presentation of the critical immunogenic epitopes of the conjugated antigens, as well as T cell epitopes of the carrier proteins, in a stepwise manner.

In another embodiment, the current invention is directed towards the optional insertion of heterogeneously derived T-cell epitopes in various carrier proteins, prior to conjugation with the antigen, for the purpose of production of conjugate vaccines and augment the final T-cell mediated response of the resultant hybrid carrier proteins.

In certain embodiments the CAPDAdjuvant nanoparticle vaccine compositions, comprising reCaP, hereof comprise aluminum as an adjuvant, e.g., in the form of aluminum hydroxide, aluminum phosphate, aluminum potassium phosphate, calcium phosphate or combinations thereof, in concentrations of 0.05-5 mg, e.g., from 0.075-1.0 mg, of aluminum content per dose.

In other aspects, the present disclosure provides a method of eliciting an immune response against the conjugated immunogen that is part of the CAPDAdjuvant nanoparticle, comprising reCaP, composition in a subject, such as a human, comprising administering to the subject an effective amount of the conjugated immunogen that is part of the CAPDAdjuvant nanoparticle composition; a nucleic acid molecule encoding the conjugated immunogen that is part of the CAPDAdjuvant nanoparticle composition; or a composition comprising the conjugated immunogen that is part of the CAPDAdjuvant nanoparticle composition or nucleic acid molecule. The present disclosure also provides a method of preventing infection in a subject, comprising administering to the subject an effective amount of a pharmaceutical composition, such as a conjugate vaccine that is part of the CAPDAdjuvant nanoparticle composition. In some particular embodiments, the pharmaceutical composition comprises a nucleotide encoding the conjugated immunogen that is part of the CAPDAdjuvant nanoparticle composition. In some embodiments of the methods provided herein above, the subject is a human. In some particular embodiments, the human is a child, such as an infant. In some other particular embodiments, the human is a woman, particularly a pregnant woman.

The effective amount administered to the subject is an amount that is sufficient to elicit an immune response against a conjugated antigen, comprising reCaP, defined by the immunogen that is part of the CAPDAdjuvant nanoparticle composition in the subject.

In addition to the immunogenic component, the CAPDAdjuvant nanoparticle vaccine compositions further comprise an immunomodulatory agent, such as an adjuvant. Examples of suitable adjuvants include aluminum salts such as aluminum hydroxide, aluminum phosphate and/or calcium phosphate; derivatives of PGA, chitosan, CpG-motif containing oligonucleotides, ADP-ribosylating bacterial toxins or mutants thereof, such as E. coli heat labile enterotoxin LT, cholera toxin CT, and the like.

The design of multimerization domains for the formation of CAPDAdjuvant nanoparticles can be identified by methods known in the art, such as by visual inspection of a crystal structure of an immunogen, or by using computational protein design software such as BioLuminate™ [BioLuminate, Schrodinger LLC, New York, 2015 ], Discovery Studio™ [Discovery Studio Modeling Environment, Accelrys, San Diego, 2015 ], MOE™ [Molecular Operating Environment, Chemical Computing Group Inc., Montreal, 2015 ,]and Rosetta™ [Rosetta, University of Washington, Seattle, 2015]). The amino acids to be utilized for the design of multimerization domains for the formation of CAPDAdjuvant nanoparticle typically include small aliphatic (e.g. Gly, Ala, and Val) or small polar amino acids (e.g. Ser and Thr). The amino acids can be large aliphatic amino acids (Ile, Leu and Met) or large aromatic amino acids (His, Phe, Tyr and Trp).

The peptide-linked carrier proteins provided by the present disclosure can be prepared by routine methods known in the art, such as by expression in a recombinant host system using a suitable vector. Suitable recombinant host cells include, for example, insect cells, mammalian cells, avian cells, bacteria, and yeast cells. Examples of suitable insect cells include, for example, Sf9 cells, Sf21 cells, Tn5 cells, Schneider S2 cells, and High Five cells (a clonal isolate derived from the parental Trichoplusia ni BTI-TN-5B1-4 cell line (Invitrogen)). Examples of suitable mammalian cells include Chinese hamster ovary (CHO) cells, human embryonic kidney cells (HEK293 or Expi 293 cells, typically transformed by sheared adenovirus type 5 DNA), NIH-3T3 cells, 293-T cells, Vero cells, and HeLa cells. Suitable avian cells include, for example, chicken embryonic stem cells (e.g., EBx™ cells), chicken embryonic fibroblasts, chicken embryonic germ cells, quail fibroblasts (e.g. ELL-O), and duck cells. Suitable insect cell expression systems, such as baculovirus-vectored systems, are known to those of skill in the art and described in, e.g., Summers and Smith, Texas Agricultural Experiment Station Bulletin No. 1555 (1987). Materials and methods for baculovirus/insect cell expression systems are commercially available in kit form from inter alia, Invitrogen, San Diego Calif. Avian cell expression systems are also known to those of skill in the art and described in, e.g., U.S. Pat . Nos. 5,340,740; 5,656,479; 5,830,510; 6,114,168; and 6,500,668. Similarly, bacterial and mammalian cell expression systems are also known in the art and described in, e.g., Yeast Genetic Engineering (Barr et al., eds., 1989) Butterworths, London.

A number of suitable vectors for expression of recombinant proteins in insect or mammalian cells are well-known and conventional in the art. Suitable vectors can contain a number of components, including, but not limited to one or more of the following: an origin of replication; a selectable marker gene; one or more expression control elements, such as a transcriptional control element (e.g., a promoter, an enhancer, a terminator), and/or one or more translation signals; and a signal sequence or leader sequence for targeting to the secretory pathway in a selected host cell (e.g., of mammalian origin or from a heterologous mammalian or non-mammalian species). For example, for expression in insect cells a suitable baculovirus expression vector, such as pFastBac (Invitrogen), is used to produce recombinant baculovirus particles. The baculovirus particles are amplified and used to infect insect cells to express recombinant protein. For expression in mammalian cells, a vector that will drive expression of the construct in the desired mammalian host cell (e.g., Chinese hamster ovary cells) is used.

The peptide-linked carrier proteins used in the CAPDAdjuvant nanoparticles can be purified using any suitable methods. For example, methods typically used for protein antigens such as immunoaffinity chromatography are known in the art. Suitable methods for purifying desired peptide-linked protein immunogens include precipitation and various types of chromatography, such as hydrophobic interaction, ion exchange, affinity, chelating and size exclusion are well-known in the art. Suitable purification schemes can be created using two or more of these or other suitable methods. If desired, the peptide-linked protein immunogens can include a “tag” that facilitates purification, such as an epitope tag or a histidine (HIS) tag. Such tagged polypeptides can conveniently be purified, for example from conditioned media, by chelating chromatography or affinity chromatography.

### EXAMPLES

The invention is further described by the following illustrative examples. The examples do not limit the invention in any way. They merely serve to clarify the invention.

### Example 1. Methods for Structural Modeling

The structure of CTB pentamer (PDB code 5ELB) was used to build fusion protein structures. This was accomplished by Phyre2 web-server (The Phyre2 web portal for protein modeling, prediction and analysis L A Kelley, S. Mezulis, C M Yates, M N Wass, M J E Sternberg, Nature Protocols, 2015, 10, 845-858). The resulting structure was placed in a dodecahedral box with dimensions such that there was 1 nm from the structure to each box edge. The box was then solvated with explicit TIP3P water, neutralized with 0.1 M excess NaCl, and subject to 1,000 steps of steepest descent energy minimization, 2 ns of NVT equilibration, 2 ns of NPT equilibration and 50 ns of NPT production using the CHARMM27 force field in GROMACS 4.6.7 (GROMACS 4: Algorithms for Highly Efficient, Load-Balanced, and Scalable Molecular Simulation. B. Hess, C. Kutzner, D. van der Spoel, E. Lindahl. Chem. Theory Comput. 2008, 4, 3, 435-447). A time step of 2 fs was used for all equilibration and production runs.

### Example 2. DNA Manipulations and Molecular Cloning

The mature sequence of Vibrio cholerae 01 biovar El Tor strain RDS866 enterotoxin subunit B (SEQ ID NO: 1) was submitted for codon-optimization (Codon Optimization Tool found at https://www.idtdna.com/CodonOpt) for expression in E. coli cells. Additional codon-optimization and gene syntheses were performed by IDT (Coralville, Iowa). All oligonucleotides were purchased from Sigma-Aldrich (St. Louis, Mo.), and restriction endonucleases were purchased from New England Biolabs (Ipswich, Mass.). DNA assembly reactions were performed with NEBuilder HiFi DNA Assembly Master Mix (New England Biolabs). pET28a (MilliporeSigma, Burlington, Mass.) expression vector was digested with Ncol-HF and BamHI-HF, and overlapping sequences to facilitate insertion of synthetic DNA fragments into the vector were added during DNA synthesis (SEQ ID NO: 3 and SEQ ID NO: 4 added to the 5′ and 3′ end, respectively). Cloning reactions were transformed into E. coli DH5a cells, and selection was performed on LB plates containing 30 μg/mL kanamycin. Plasmid purifications were performed with the Wizard Plus SV Minipreps DNA Purification System (Promega, Madison, Wis.). The presence of the intended nucleotide sequence was confirmed by DNA sequencing (Eurofins, Louisville, Ky.).

### Example 3: Recombinant CTB Fusion Protein (MCFP) Expression

E. coli BL21 (DE3) was used as the expression host. Cells harboring expression plasmids were cultured overnight in LB medium containing 50 μg/mL kanamycin. The following day, cells were inoculated into 2X YT medium to a starting optical density at 600 nm (0D600) between 0.005 and 0.02. The cultures were incubated at 37° C. with shaking at 250 rpm. When the cultures reached OD600 values around 0.6 to 0.8, a pre-induction sample (t=0) was collected and frozen at −20° C., and IPTG was added to the remaining culture at a final concentration of 1 mM. The cultures were incubated for an additional 3 hours at 37° C. with shaking at 250 rpm. Following this incubation, cell samples were harvested (t=3) and frozen at −20° C.

### Example 4. Recombinant CTB Fusion Protein (MCFB) Purification

Recombinant CTB fusion proteins were purified by IMAC in batch mode. Frozen cell pellets were resuspended in 1×BugBuster Protein Extraction Reagent (MilliporeSigma) containing 100 mM Tris, pH 7.9, 100 mM NaCl, 5 mM imidazole, Benzonase (MilliporeSigma), and cOmplete EDTA-free Protease Inhibitor (Roche, Indianapolis, Ind.). Cells were lysed at room temperature for 20 min, and insoluble material was removed by centrifugation. The soluble protein fraction was incubated with equilibrated HisPur Ni-NTA Resin (Thermo Fisher Scientific, Pittsburgh, Pa.), and the resin was washed with buffers containing increasing concentrations of imidazole. Following the washes, the recombinant protein was eluted in buffer containing 20 mM Tris-HC1, pH 7.9, 0.5 M NaCl, 1 M imidazole. Protein quantification was performed with the Quick Start Bradford Protein Assay (Bio-Rad, Hercules, Calif.) as described for the Standard assay performed in microplate format.

### Example 5. Periplasmic Localization

Periplasmic localization of expressed recombinant CTB fusion protein was assessed by a method outlined previously (Neu HC, Heppel La. The release of enzymes from Escherichia coli by osmotic shock and during the formation of spheroplasts. J Biol Chem. 1965; 240(9):3685-3692.)

### Example 6. GM1 ELISA

ELISA for CTB proteins binding to GM1 ganglioside was performed in a similar manner as described previously (Liao J, Gibson J A, Pickering B S, Watnick P I. 2018. Sublingual adjuvant delivery by a live attenuated Vibrio cholerae-based antigen presentation platform. mSphere 3:e00245-18.) Wells of a Nunc Maxisorp (Thermo Fisher Scientific) plate were coated with 100 ng GM1 ganglioside (AdipoGen Life Sciences, San Diego, Calif.) prepared in 50 mM sodium carbonate buffer, pH 9.6. 50 mM sodium carbonate buffer, pH 9.6, was added to non-coated wells. The plate was covered and incubated overnight at room temperature. After the solution was decanted, 100 uL of a 1% solution of bovine serum albumin (BSA) prepared in 50 mM sodium carbonate buffer, pH 9.6 was added to each well to block, and the plate was incubated for 2 hours at room temperature. This solution was decanted, and the wells were washed twice with 100 uL 1×phosphate-buffered saline (PBS), pH 7.4, containing 0.1% Tween 20 (PBS-T). 100 uL of the following concentrations of either CTRNVS, commercially available CTB (sourced from Sigma-Aldrich, protein referred to as Sigma CTB), or BSA were added to the wells: 0.5 ug/mL, 0.25 ug/mL, 0.125 ug/mL, 0.0625 ug/mL, 0.03125 ug/mL, 0.015625 ug/mL, 0.007813 ug/mL, 0.003906 ug/mL, 0.001953 ug/mL, 0.000977 ug/mL, 0.000488 ug/mL. Proteins were diluted in 1×PBS, and a column of wells where no protein was added was included as a blank. The plate was covered and incubated overnight at room temperature. This solution was decanted, and the wells were washed twice with 100 uL PBS-T. The plate was further blocked with 100 uL PBS-T+1% BSA. The plate was incubated for 2 hours at room temperature. This solution was decanted, and 100 uL of a 1 ug/mL solution (diluted in PBS-T+1% BSA) of α-CTB mouse monoclonal antibody (Sigma-Aldrich) was added to each well. The plate was incubated for 2 hours at room temperature. This solution was decanted, and the wells were washed twice with 100 uL PBS-T. 100 uL of a 1:6,000 dilution (made in PBS-T+1% BSA) of Goat anti-Mouse IgG, Human adsorbed, HRP-conjugated polyclonal antibody (Southern Biotech, Birmingham, Ala.) was added to each well. The plate was incubated for 30-60 minutes at room temperature. This solution was decanted, and the wells were washed twice with 100 uL PBS-T. 100 uL of 1×TMB solution (Thermo Fisher Scientific) was added to each well. The plate was incubated at room temperature until a faint blue color developed in some of the wells. At this point, the reaction was stopped by addition of 100 uL 0.18 M H2SO4 to all of the wells. The plate was read on a spectrophotometer set to read absorbance at 450 nm. Absorbance values for each sample were plotted following subtraction of the absorbance values obtained from the blank wells.

### Example 7: Western Blot Hybridization

Following electrophoretic separation, proteins were transferred to PVDF membrane. All membrane blocking and antibody hybridization steps were performed in Tris-buffered saline containing 0.1% Tween 20 (TBS-T) and 5% non-fat dry milk. All membrane wash steps were performed in TBS-T. The membrane was probed with a mouse monoclonal anti-cholera toxin B subunit-specific antibody (Sigma-Aldrich) diluted to 1 μg/mL. Goat anti-mouse IgG (H+L)-AP Conjugate (Thermo Fisher Scientific) at a 1:10,000 dilution was used as the secondary antibody. Detection was performed with the AP Conjugate Substrate Kit (Bio-Rad). Western blots were processed using standard protocols (Gallagher S, Winston S, Fuller S, Hurrell J. 2008. Immunoblotting and Immunodetection, p 10.8.1-10.8.28, Current Protocols in Molecular Biology, vol 83. John Wiley & Sons, Inc.)

### Example 8: Design and Preparation of SAANPs Containing the Carrier Proteinn Cholera Toxin B (CTB)

This example outlines the design and preparation of self-assembling nanoparticles containing CTB, which includes (i) a linker peptide and/or a T-cell epitope optionally without lysine residues; (ii) peptide antigen and (iii) DFP. In step one, modified CTB carrier proteins (MCFPs) are produced with covalently linked with peptides (i), (ii) and (iii). mixed. In optional step 2, the MCFP is chemically coupled with haptens or peptide or carbohydrate antigens to produce conjugated antigens; In step 3, the MCFP produced in step 2 or conjugate produced in step 3 is formulated by mixing with the adjuvant, such as PGA and is non-covalently complexed together to produce SAANP vaccine system.

### REFERENCES

Newman et al., Immunological and Formulation Design Considerations for Subunit Vaccines in “Vaccine Design: The Subunit and Adjuvant Approach” (Powell and Newman, Editors), Plenum Press, New York, 1995, pp 1-42.

Prasad, A. Krishna. Introduction in “Carbohydrate-based vaccines: from concept to clinic” (Prasad, A. Krishna, Editor), American Chemical Society Symposium series, 2018, vol 1290, pp 1-6.

Svenson, S. B. et al., Infection and Immunity, 1979, 863-872.

Dintzis, R. Z.; Pediatr Res., 1992, 32 (4): 376-385.

Tam JP: Synthetic peptide vaccine design: synthesis and properties of a high-density multiple antigenic peptide system. Proc Natl Acad Sci USA. 1988, 85 (15): 5409-5413.

Posnett et al., Multiple Antigenic Peptide Method for producing antipeptide site-specific antibodies. Methods Enzymol. 1989; 178: 739-746.

Posnett et al., A novel method for producing anti-peptide antibodies. Production of site-specific antibodies to the T cell antigen receptor beta-chain. 1988; 263 (4): 1719-1725.

Fujita et al., Current status of multiple antigen-presenting peptide vaccine systems: Application of organic and inorganic nanoparticles. Chemistry Central Journal, 2011, 5:48; 1-8.

Moyer et al., Beyond antigens and adjuvants. J. Clin. Invest. 2016; 126 (3), 799-808.

Wille-Reece, et al. HIV Gag protein conjugated to a Toll-like receptor 7/8 agonist improves the magnitude and quality of Th1 and CD8+ T cell responses in nonhuman primates. Proc Natl Acad Sci U S A. 2005

de Titta A, et al. Nanoparticle conjugation of CpG enhances adjuvancy for cellular immunity and memory recall at low dose. Proc Natl Acad Sci U S A. 2013;110(49):19902-19907.

Moon J J, et al. Interbilayer-crosslinked multilamellar vesicles as synthetic vaccines for potent humoral and cellular immune responses. Nat Mater. 2011;10(3):243-251.

Ilyinskii, et al. Adjuvant-carrying synthetic vaccine particles augment the immune response to encapsulated antigen and exhibit strong local immune activation without inducing systemic cytokine release. Vaccine. 2014.

Cimica, V., et al., “Novel Respiratory Syncytial Virus-Like Particle (VLP) Vaccine Composed of the Postfusion and Prefusion Conformations of the F Glycoprotein”, Clin. Vaccine Immunol, 2016, pp. 1-33.

Donaldson et al., Virus-like particle vaccines: immunology and formulation for clinical translation. Expert Rev. Vaccines, 2018; 17 (9): 833-849.

Silva et al., PLGA particulate delivery systems for subunit vaccines: Linking particle properties to immunogenicity. Hum Vaccin Immunother. 2016; 12 (4): 1056-1069.

Reed et al., Key roles of adjuvants in modern vaccines. Nature Medicine. 2013; 19: 1597-1608.

Noh, H. J. et al., Programming of Influenza Vaccine Broadness and Persistence by Mucoadhesive Polymer-Based Adjuvant Systems J Immunol 2015; 195: 2472-2482

Buescher, J. M., and A. Margaritis. Microbial biosynthesis of polyglutamicacid biopolymer and applications in the biopharmaceutical, biomedical and food industries. Crit. Rev. Biotechnol. 2007; 27: 1-19.

Kim, T. W., T. Y. Lee, H. C. Bae, J. H. Hahm, Y. H. Kim, C. Park, T. H. Kang, C. J. Kim, M. H. Sung, and H. Poo. Oral administration of high molecular mass poly-g-glutamate induces NK cell-mediated antitumor immunity. J. Immunol., 2007, 179: 775-780.

Rosano, G L, Ceccarelli, E E: Recombinant protein expression in Escherichia coli: advances and challenges. Front. Microbiol. 2014, 5, 172, 1-17.

Walls, A. C, Park, Y. J., Tortorici, M. A., Wall, A., McGuire, A. T., Veesler, D., Cell, 2020, 180, 281-292.

Slos P, Speck D, Accart N, et al. Recombinant cholera toxin B subunit in Escherichia coli: high-level secretion, purification, and characterization. Protein Expr Purif. 1994;5(5):518-526.

Sánchez J, Holmgren J. Cholera toxin structure, gene regulation and pathophysiological and immunological aspects. Cell Mol Life Sci. 2008; 65(9):1347-1360.

Hartgerink J D, Granj a J R, Milligan R A, Ghadiri M R: Self-assembling peptide nanotubes. J Am Chem Soc. 1996, 118 (1): 43-50.

Rajagopal K, Schneider J P: Self-assembling peptides and proteins for nanotechnological applications. Curr Opin Struct Biol. 2004, 14 (4): 480-486.

Harbury et al., “A switch between two-, three-, and four-stranded coiled coils in GCN4 Leucine Zipper Mutants,” Science,1993,1401-1407, vol. 262.

Hoppe et al., “A parallel three stranded a-helical bundle at the nucleation site of collagen triple-helix formation”, FEBS Letters, 1944,191-195, vol. 344.

Lopez-Sagaseta et al., Self-assembling protein nanoparticles in the design of vaccines. Computational and Structural Biotechnology Journal, 2016, 14: 58-68.

McAlinden et al., “a-Helical Coiled-coil Oligomerization Domains Are Almost Ubiquitous in the Collagen Superfamily,” Biological Chemistry, 2003, 42200-42207, vol. 278.

Miroshnikov et al., “Engineering trimeric fibrous proteins based on bacteriophage T4 adhesins”, Protein Eng Des Sel, 1998 11:329-414.

Todd et al., “Designing supramolecular protein assemblies”, Current Opinion in Structural Biology, 2002, 12, pp. 464-470.

Jung et al., Co-assembling peptides as defined matrices for endothelial cells. Biomaterials. 2009, 30 (12): 2400-2410.

Rudra et al., A self-assembling peptide acting as an immune adjuvant. Proc Natl Acad Sci USA. 2010, 107 (2): 622-627.

Burkhard et al., “Coiled coils: a highly versatile protein folding motif”, Trends in Cell Biology, vol. 11, No. 2, pp. 82-88, Feb. 2001.

Pimentel et al., Peptide nanoparticles as novel immunogens: design and analysis of a prototypic severe acute respiratory syndrome vaccine. Chem Biol Drug Des. 2009, 73 (1): 53-61.

Kaba et al., A nonadjuvanted polypeptide nanoparticle vaccine confers long-lasting protection against rodent malaria. J Immunol. 2009, 183 (11): 7268-7277.

Lapelosa et al., n silico vaccine design based on molecular simulations of rhinovirus chimeras presenting HIV-1 gp41 epitopes. J Mol. Biol. 2009; 385 (2): 675-691.

McLellan, et al., “Structure of RSV Fusion Glycoprotein Trimer Bound to a Prefusion-Specific Neutralizing Antibody”, Science, 2013, 1113-1117, vol. 309.

Prisco et al., Filamentous Bacteriophage Fd as an Antigen Delivery System in Vaccination. Int. J. Molec. Sci. 2012; 13 (4): 5179-5194.

Mahmoudi et al., Efficacy of phase 3 trial of RTS, S/AS01 malaria vaccine: The need for an alternative development plan. Hum. Vaccin. Immunother. 2017; 13 (9); 2098-2106.

Melero, J., “Influence of antigen conformation and mode of presentation on the anitbody and protective responses against human respiratory syncytial virus: relevance for vaccine development,” Expert Review of Vaccines, 2016, pp. 1-8.

Graham et al., Structure-based Vaccine Antigen Design. Annu. Rev. Med, 2019; 70: 91-104.

Kanekiyo et al., Self-assembling influenza nanoparticle vaccines elicit broadly neutralizing H1N1 antibodies. Nature. 2013; 499(7456): 102-106.

Darricarrere et al., J. Virol., 2018, 92 (22), e01349-18.

National Institutes of Health News, 2019, 4 Apr. https://www.nih.gov/inews-events/news-releases/nih-begins-first-human-trial-universal-influenza-vaccine-candidate. Accessed on 24 Apr. 2019.

Pichichero, M. E. et al., Protein carriers of conjugate vaccines. Hum Vaccin Immunotherap, 2013, 9 (12): 2505-2523.

Avci, F., Berti, F., Dull, P., Hennessey, J., Pavliak, V., Prasad, A. K., Vann, W., g Wacker, M., Marcq, 0., Glycoconjugates: What It Would Take To Master These Well-Known yet Little-Understood Immunogens for Vaccine Development, mSphere, 2019, 4 (5) e00520-19.

Pollabauer, E. M. et al., Vaccine, 2009 Mar 10; 27(11):1674-1679.

HogenEsch et al., Optimizing the utilization of aluminum adjuvants in vaccines: you might just get what you want. NPJ Vaccines, 2018, 51, 1-11.

Li et al., Interfacial stress in the development of biologics: fundamental understanding, current practice and future perspective. AAPS J; 2019; 21(44): 1-17.

Chen, X, Zaro, J, and Shen, W-C., Fusion Protein Linkers: Property, Design and Functionality, Adv Drug Deliv Rev. 2013 Oct. 15; 65 (10): 1357-1369.

Bixler, G. S., et al., In Immunobiology of Proteins and Peptides V; Atassi, M. Z., Ed.; Springer Science+Business Media: New York, 1989; 175-180.

Bixler, G. S. et al., U.S. Pat. No. 5,785,973, 28 Jul. 1998.

Micoli, F. et al., Protein Carriers for Glycoconjugate Vaccines: History, Selection Criteria, Characterization and New Trends. Molecules, 2018, 23 (6), 1-18.

He L M, Neu M P and Vanderberg L A: “Bacillus licheniformis g-glutamyl exopolymer: physicochemical characterization and UV interaction,” Environ Sci Technol. 2000, 34: 1694-1701.

Madore D V et al., Immune response of young children vaccinated with Haemophilus influenzae type b conjugate vaccines. Contrib Microbiol Immunol. 1989; 10: 125-150.

Baldauf, K. J., Cholera Toxin B: One Subunit with Many Pharmaceutical Applications. Toxins, 2015, 7, 974-996.

Harakuni T, et al., Heteropentameric cholera toxin B subunit chimeric molecules genetically fused to a vaccine antigen induces systemic and mucosal immune responses: a potential new strategy to target recombinant vaccine antigens to mucosal immune systems. Infect Immun. 2005 September; 73(9):5654-65.

Bergerot I, et al., A cholera toxoid-insulin conjugate as an oral vaccine against 486 spontaneous autoimmune diabetes. Proc Natl Acad Sci USA. 1997; 94(9): 4610-4614.

Verweij W R, de Haan L, Holtrop M, Agsteribbe E, Brands R, van Scharrenburg G J M, Wilschut J. Vaccine. 1998; 16:2069-2076.

1. A composition, comprising a plurality of components bound to an adjuvant core, the components comprising:
a recombinant carrier protein, expressed as a multifunctional chimeric fusion protein (MCFP), covalently linked to a linker peptide and/or, optionally, a T-cell epitope and/or a pathogen-specific epitope;
a linker peptide and/or, optionally, a T-cell epitope covalently linked with the carrier protein;
a pathogen-specific peptide antigen covalently linked to the carrier protein and/or linker peptide and/or, optionally, a T-cell epitope; and
a terminal dual function peptide covalently linked directly to the carrier protein or indirectly via the linker or T-cell epitope or the antigen, capable of binding to the adjuvant and blocking core adjuvant surface space from non-specific binding, thereby controlling density of the peptide antigen (and the carrier protein linked to the peptide antigen and/or linker) capable of forming a nanoparticle.

2. The composition of claim 1, wherein the dual function peptide is a terminal dual function peptide.

3. The composition of claim 1 or 2, wherein the pathogen-specific peptide antigen includes a polypeptide antigen from the Receptor Binding Domain (RBD) of SARS-CoV-2 spike protein.

4. The composition of claim 3, including SEQ ID NO: 36, 40, 43 or 46.

5. An integrated Multiple Antigen displayed Adjuvant System (iMAAS), comprising a Self Assembling Adjuvanted Nanoparticle (SAANP) comprising a plurality of components that are bound to an adjuvant core; wherein:
(i) a first component of said plurality comprises a recombinant carrier protein capable of forming a self-assembling nanoparticle;
(ii) a second component of said plurality is a linker peptide and/or, optionally, a T-cell epitope covalently linked with the carrier protein and/or a peptide antigen;
(iii) a third component of said plurality comprises a peptide antigen covalently linked to the carrier protein and/or linker peptide, which is a pathogen-specific antigen; and
(iv) a fourth component of said plurality comprises a terminal dual function peptide, covalently linked to the linker or, optionally, T-cell epitope or the peptide antigen, capable of binding to the adjuvant and blocking core adjuvant surface space from non-specific binding, thereby controlling the density of the peptide antigen (and the carrier protein linked to the peptide antigen) capable of forming a nanoparticle.

6. The integrated Multiple Antigen displayed Adjuvant System (iMAAS) of claim 5, wherein the Multifunctional Chimeric Fusion Protein is produced by recombinant protein expression in Escherichia coli.

7. The integrated Multiple Antigen displayed Adjuvant System (iMAAS) of claim 5 or 6, wherein the terminal DFP portion is bound to the adjuvant via the formation of adjuvanted nanoparticles.

8. The integrated Multiple Antigen displayed Adjuvant System (iMAAS) of any one of claims 5-7, wherein the carrier protein comprises recombinantly expressed Cholera Toxin B.

9. The integrated Multiple Antigen displayed Adjuvant System (iMAAS) of any one of claims 5-8, comprising SEQ ID NO: 25.

10. The integrated Multiple Antigen displayed Adjuvant System (iMAAS) of any one of claims 5-9, comprising a recombinantly expressed Cholera Toxin B linked to a terminal dual function peptide capable of acting as a protein purification aid and having the affinity to non-covalently bind to adjuvants.

11. The integrated Multiple Antigen displayed Adjuvant System (iMAAS) of any one of claims 5-10, wherein the dual function peptide comprises a polyhistidine tag having 6-10 contiguous histidine residues.

12. The integrated Multiple Antigen displayed Adjuvant System (iMAAS) of any one of claims 5-11, comprising a carrier protein that includes recombinantly expressed Cholera Toxin B, incorporating peptide antigens derived from SARS CoV-2 virus spike protein S.

13. The integrated Multiple Antigen displayed Adjuvant System (iMAAS) of claim 12, wherein the recombinantly expressed Cholera Toxin B, incorporating peptide antigens derived from SARS CoV-2 virus spike protein S, comprises SEQ ID NOs 36-54, or sequences at least 85, 86, 87, 88, 89, 90, 91, 92, 93, 94, 95, 96, 97, 98 or 99% identical to SEQ ID NOs 36-54.

14. The integrated Multiple Antigen displayed Adjuvant System (iMAAS) of claim 13, comprising a recombinantly expressed Cholera Toxin B, incorporating peptide antigens derived from SARS CoV-2 virus spike protein S, having SEQ ID NO: 43.

15. The integrated Multiple Antigen displayed Adjuvant System (iMAAS) of claim 13, comprising a recombinantly expressed Cholera Toxin B, incorporating peptide antigens derived from SARS CoV-2 virus spike protein S, having SEQ ID NO: 46.

16. The integrated Multiple Antigen displayed Adjuvant System (iMAAS) of any one of claims 5-15, comprising a carrier protein that is recombinantly expressed Cholera Toxin B, incorporating heterologously derived T-cell peptides without lysine residues, from a different carrier protein.

17. The integrated Multiple Antigen displayed Adjuvant System (iMAAS) of any one of claims 5-16, comprising a recombinantly expressed carrier protein, incorporating heterogeneously derived T-cell peptides without lysine residues, from a different carrier protein.

18. The integrated Multiple Antigen displayed Adjuvant System (iMAAS) of any one of claims 5-17, wherein the adjuvanted system iMAAS, better stimulates an innate immune response and/or has improved stability of the antigen, compared to a soluble monomeric form of a vaccine antigen.

19. The iMAAS of any one of claims 5-18, wherein the carrier protein, the linker peptide and DFP comprises SEQ ID NO: 25, or sequences at least 85, 86, 87, 88, 89, 90, 91, 92, 93, 94, 95, 96, 97, 98 or 99% identical to SEQ ID NO: 25.

20. The iMAAS of any one of claims 5-19, wherein the carrier protein, the linker peptide, the T cell epitope and DFP includes SEQ ID NOs: 31 or 35, or sequences at least 85, 86, 87, 88, 89, 90, 91, 92, 93, 94, 95, 96, 97, 98 or 99% identical to SEQ ID NOs: 31 or 35.

21. The iMAAS of any one of claims 5-20, wherein:
the carrier protein includes one of SEQ ID NOs: 6, 8, 21, 23 and 25;
the linker peptide and/or T-cell epitope includes one of SEQ ID NOs: 6, 8, 21, 23, 25, 31, 35, 40, 43, 46, 50 and 54;
the pathogen-specific peptide antigen includes one of SEQ ID NOs: 31, 35, 40, 43, 46, 50 and 54; and/or the dual function peptide includes one of SEQ ID NOs: 6, 8, 21, 23, 25, 31, 35, 40, 43, 46, 50 and 54;
wherein any of the above-listed sequences are at least 85, 86, 87, 88, 89, 90, 91, 92, 93, 94, 95, 96, 97, 98, 99 or 100% identical to the above-listed Sequence Identifiers.

22. The iMAAS of any one of claims 5-21, comprising SEQ ID NO: 43 or a sequence at least 85, 86, 87, 88, 89, 90, 91, 92, 93, 94, 95, 96, 97, 98 or 99% identical to SEQ ID NO: 43.

23. The iMAAS of any one of claims 5-21, comprising SEQ ID NO: 46 or a sequence at least 85, 86, 87, 88, 89, 90, 91, 92, 93, 94, 95, 96, 97, 98 or 99% identical to SEQ ID NO: 46.

24. The iMAAS of any one of claims 5-23, comprising CTB and the peptide antigen from the RBD of SARS CoV-2 spike protein S.

25. The integrated Multiple Antigen displayed Adjuvant System (iMAAS) of any one of claims 5-24, wherein the adjuvant is ionically charged.

26. The integrated Multiple Antigen displayed Adjuvant System (iMAAS) of any one of claims 5-24, wherein the adjuvant is a polyglutamic acid or modified polyglutamic acid.

27. The integrated Multiple Antigen displayed Adjuvant System (iMAAS) of any one of claims 5-26, comprising a carrier protein that includes recombinantly expressed Cholera Toxin B, incorporating peptide antigens derived from SARS CoV-2 virus spike protein S as a prophylactic vaccine to target COVID-19 disease.
